(19) United States
(12) Patent Application Publication
(10) Pub. No.: US 2005/0015140 A1
(43) Pub. Date: Jan. 20, 2005

(54) Encapsulation device and methods of use

(76) Inventor: Nicholas deBeer, Montara, CA (US)

Correspondence Address: NICHOLAS DeBEER, 512 7TH STREET, P.O. BOX 371373, MONTARA, CA, 94037, US

(21) Appl. No.: 10/621,730

(22) Filed: Jul. 14, 2003

(51) Int. Cl. A61F 2/06; A61F 2/44; A61F 2/04

(52) U.S. Cl. 623/1.21; 623/1.36; 623/23.67; 623/17.12

(57) ABSTRACT

An encapsulation device having an expandable, porous body with a cavity, the body having a sealed end and a sealable end, where the body is configured to receive one or more fluids through a port in the sealable end, the body is configured to expand to conform to a shape of a target, and where the sealable end may be sealed to prevent leakage into the body, and a method if using the encapsulation device, is disclosed. The body may comprise porous membrane including expanded Polytetrafluoroethylene (ePTFE), polyester fiberfill, metal/polymer mesh, and perforated or porous polymer/metal. The target may include a location within the human body. The port in the sealable end is configured to receive a first fluid into the cavity to expand the body to conform to the shape of the target, and the port is further configured to receive a second fluid into the cavity which displaces the first fluid by diffusing the first fluid through the pores in the body and which cures (such as, by an ultraviolet light source) to secure the body to the target. Optionally, the introduction of the second fluid may be unnecessary when the first fluid includes the desired filler material for the encapsulation device.

### BACKGROUND OF THE INVENTION

The present invention relates to an application for three-dimensional space filling encapsulation devices which may be filled with a bioactive agent or anchored in vivo. More specifically, the present invention relates to applications for encapsulation through filling a member directly or through diffusion, displacement of one or more fluids, and the replacement of the fluids with one or more other fluids or hydrogels. Wire assisted membrane shape formation may aid delivery or final dimension of the encapsulation.

It is difficult to deploy complex shapes through small portals in the body of an animal. For many body conditions, it is often the case that invasive surgical procedures are necessitated due to the fact that isolation and application of a target treatment is often difficult given the complexity of the portals in the animal body. Use of stents and the like are commonplace for deploying medication and so on. However, existing approaches have limitations due to the shortcomings of the stents, whether medically coated or not.

Moreover, because of the complexity of the shapes in the various areas of the animal body, one challenge is to provide a three dimensional structure which may be easily deployed to the target area of the body, and provide structural integrity at the desired site, whether for physical support or for controlled release of a particular medication. Additionally, there are many biocompatible and bioactive adhesives and non-adhesive materials that can change shape from liquid to solid or semi-solid in the body. Further, there are bioactive materials such as poly glycolic and lactic acid (PGLA) which can be added to the body to take advantage of its properties. But there are few ways of providing safe containment of such materials in the body. Many materials can be made to elude drugs but need to be more isolated from the body. Furthermore, many devices need a way of being anchored once they are deployed to the desired location within the body especially in case of using bioactive agents to achieve the desired therapeutic results.

In view of the foregoing, it is desired to have a three dimensional encapsulation device which may be deployed in vivo and that provides structural integrity by, for example, anchoring itself to the walls of the target site, and whose physical dimension provides flexibility in size and complexity for a broad application for the many desired target sites.

### SUMMARY OF THE INVENTION

In one embodiment of the present invention, there is provided an encapsulation device which includes an expandable, porous body having a cavity therein, the body having a sealed end and a sealable end, where the body is configured to receive one or more fluids through a port in the sealable end, where the body is configured to expand to conform to a shape of a target, and where the sealable end may be sealed to prevent leakage into the body.

The body may comprise expanded Polytetrafluoroethylene (ePTFE), and the target may comprise a location within the human body. In accordance with the various embodiments of the present invention, the body of the encapsulation device may include other materials such as porous PET or porous polyurethane, and metal such as Nitinol which is made porous by, for example, laser drilled holes thereon, or other standard methods of making Nitinol porous.

Moreover, the port in the sealable end in one embodiment may be configured to receive a first fluid into said cavity to expand the body to conform to the shape of the target, and the port may further be configured to receive a second fluid into the cavity which displaces the first fluid by diffusing the first fluid through the pores in the body and which cures to secure the body to the target.

The first fluid may comprise a saline solution, while the second fluid may comprise an adhesive. The second fluid may be more or less viscous than the first fluid. For example, in one embodiment, a two-part hydrogel comprised on one part Polyvinylpyrrolidone (PVP) and one part Polyethyleneimine (PEI) may be used as the first and the second fluids, such that when mixed together, the resulting hydrogel exhibits cohesive and elastic properties. In this case, the first fluid PVP is more viscous than the second fluid PEI, and thus, when the PVP is first injected into the encapsulation body, it tends to weep out less through the pores of the encapsulation body as compared to the second fluid PEI which is comparatively less viscous. Thereafter, upon subsequent introduction of the second fluid PEI in the encapsulation body, the resulting hydrogel is formed within the encapsulation body, taking the shape of the inner cavity thereof.

Further, the body may comprise metal having holes, where the metal may comprise nitinol.

The port in the sealable end may comprise a valve configured to open to receive one or more fluids and close to prevent leakage into the body.

Moreover, the body may include a wire reinforcement, where the wire reinforcement may comprise one or more of nitinol, stainless steel, and a structural polymer. Additionally, the wire reinforcement may be provided within the body. In a further aspect, the body may include a tubular support structure provided on the outside of the body to give reinforcement to the encapsulation body.

In one aspect, the body may comprise a first membrane and a second membrane within the first membrane, where the second membrane is more or less porous than the first membrane. Alternatively, the body may comprise a first membrane and a second membrane within the first membrane, where the first membrane is more or less porous than the second membrane.

An encapsulation device in accordance with another embodiment of the present invention includes an expandable, porous body having a cavity therein, the body having a sealed end and a sealable end, where the body is configured to receive one or more fluids through a port in the sealable end, where the body is configured to expand to conform to a shape of a target, where the sealable end may be sealed to prevent leakage into the body, where the body comprises porous membrane such as expanded Polytetrafluoroethylene (ePTFE), and further, where the port in the sealable end receives a first fluid into said cavity to expand the body to conform to the shape of the target, and wherein the port receives a second fluid into said cavity which displaces the first fluid by diffusing the first fluid through the pores in the body and which cures to secure the body to the target.

The target may comprise a location within the human body. The first fluid may comprise a saline solution, while the second fluid may comprise an adhesive.

Further, the second fluid may be more viscous than the first fluid.

Additionally, the body may comprise metal having holes, where the metal may comprise Nickel Titanium alloy (Nitinol).

The port in the sealable end may comprise a valve configured to open to receive one or more fluids and close to prevent leakage into the body.

Also, the body may comprise a wire reinforcement, where the wire reinforcement may comprise one or more of Nitinol, stainless steel, and a structural polymer. Moreover, the wire reinforcement may be provided within the body.

The body may also comprise a first membrane and a second membrane within the first membrane, where the second membrane is more porous than the first membrane. Alternatively, the body may comprise a first membrane and a second membrane within the first membrane, where the first membrane is more porous than the second membrane.

A method of providing an encapsulation device to a desired location in accordance with still another embodiment of the present invention includes expanding a porous body to conform to a shape of a target by introducing a first fluid into an opening in the body, introducing a second fluid into the porous body to displace the first fluid through the porous body, and allowing the second fluid to cure to secure the porous body to the target. In one embodiment, either the first fluid or the second fluid may be radiopaque to allow visibility under X-ray or fluoroscopy to assist, guide or monitor the deployment of the encapsulation device.

The method may further include one of the step of inserting a wire reinforcement into the porous body, the step of securing the wire reinforcement to the interior of the porous body, and the step of removing the wire reinforcement from the porous body.

The body may comprise one or more of expanded Polytetrafluoroethylene (ePTFE), porous Polyethylene Terephthalate (PET), and metal with holes formed therein.

The method may further include the step of introducing the body to a location of the target.

Also, the target may comprise a location within the human body, and where the first fluid may comprise a saline solution, while the second fluid or structural hydrogel may comprise an adhesive. In addition, the second fluid may be more viscous than the first fluid.

These and other features and advantages of the present invention will be understood upon consideration of the following detailed description of the invention and the accompanying drawings.

### BRIEF DESCRIPTION OF THE DRAWINGS

FIG. 1 is a three-dimensional perspective view of the encapsulation device shown as an empty tube in one embodiment;

FIG. 2 is a three-dimensional perspective view of the encapsulation device shown in FIG. 1 with a sealed end and a twisted end in one embodiment;

FIG. 3 is a three-dimensional perspective view of the encapsulation device shown in FIG. 1 with an end cap portion substantially cut off from the structure wall in one embodiment;

FIG. 4 is a three-dimensional perspective view of the end cap completely cut off from the encapsulation device in one embodiment;

FIG. 5 is a three-dimensional perspective view of the encapsulation device shown as a tube with a pinched end in one embodiment;

FIG. 6 is a three-dimensional perspective view of the ePTFE encapsulation device with an injection port and a sealed end in one embodiment;

FIG. 7 is a three-dimensional perspective view of the ePTFE encapsulation device of FIG. 6 with wire reinforcement in one embodiment;

FIGS. 8A-8B are a three-dimensional perspective views of the ePTFE encapsulation device NiTi wire reinforcement in one embodiment;

FIG. 9 is a three-dimensional perspective view of the ePTFE encapsulation device with displacement fluid in one embodiment;

FIG. 10 is a three-dimensional perspective view of the ePTFE encapsulation device illustrating the fluid weeping out of the pores of the ePTFE in one embodiment;

FIG. 11 is a three-dimensional perspective view of a circular tube shaped ePTFE encapsulation device in one embodiment;

FIG. 12 is a three-dimensional perspective view of a rectangular tube shaped ePTFE encapsulation device in one embodiment;

FIG. 13 is a three-dimensional perspective view of the ePTFE encapsulation device with two bonded ePTFE sheets in one embodiment;

FIG. 14 is a side view of the ePTFE encapsulation device shown in FIG. 13;

FIG. 15 illustrates a nitinol tube encapsulation device with holes laser drilled to provide porosity in accordance with one embodiment;

FIGS. 16A-16B illustrate an AAA device shown with the encapsulation seal uninflated and inflated, respectively, in accordance with one embodiment;

FIGS. 17A-17C illustrate the AAA device with the encapsulation seals formed by a flat tube, a round tube, and one ePTFE sheet, respectively, which is bonded to the outer surface of the prosthesis (stent) in accordance with one embodiment;

FIG. 18A illustrates a prosthesis with an encapsulation seal for use as an AAA device in one embodiment;

FIG. 18B illustrates the prosthesis of FIG. 18A inside an aortic aneurysm showing potential endo-leaks in accordance with one embodiment;

FIG. 19 illustrates the prosthesis of FIG. 18A inside the aortic aneurysm with encapsulation seal filler catheter attached to the injection port of the seal in accordance with one embodiment;

FIG. 20A illustrates the prosthesis of FIG. 18A with the encapsulation seal filled and in place to prevent endo-leaks in accordance with one embodiment;

FIG. 20B illustrates a cross-sectional view of the prosthesis along the line X shown in FIG. 20A in accordance with one embodiment;

FIG. 21A illustrates a prosthesis with an encapsulation seal for use as an AAA device in an aneurysm with side branch arteries in accordance with one embodiment;

FIG. 21B illustrates cross-sectional view of the prosthesis with the encapsulation seal along the line X shown in FIG. 21A in accordance with one embodiment;

FIG. 22 illustrates an encapsulation device using ePTFE tube with one sealed end and one injection port for use to occlude a lumen and used in a contraceptive application in accordance with one embodiment;

FIG. 23 illustrates the encapsulation device of FIG. 22 with a plurality of ribs in accordance with another embodiment;

FIG. 24 illustrates a two-lumen catheter for use with the encapsulation device shown in FIG. 22 in one embodiment;

FIG. 25 illustrates the encapsulation device used as a contraceptive device in accordance with one embodiment;

FIG. 26 illustrates the encapsulation device shown in FIG. 24 after the catheter is removed in accordance with one embodiment;

FIG. 27 is an ePTFE encapsulation device for use as an atrial appendage device with a separate injection port in accordance with one embodiment;

FIG. 28 is the ePTFE encapsulation device shown in FIG. 27 with wire reinforcement in accordance with one embodiment;

FIG. 29 is an encapsulation device for use as an atrial appendage device in accordance with another embodiment;

FIG. 30 illustrates a guide catheter in the left atrium of a heart for use with the atrial appendage encapsulation device of FIG. 27 in accordance with one embodiment;

FIG. 31 illustrates an ePTFE encapsulation seal with an injection port advanced through the guide catheter shown in FIG. 30 in accordance with one embodiment;

FIG. 32 illustrates the ePTFE encapsulation seal of FIG. 31 filled with fluid through the injection port via the guide catheter in accordance with one embodiment;

FIG. 33 illustrates the ePTFE encapsulation seal of FIG. 33 permanently placed at the target location with the guide catheter removed in accordance with one embodiment;

FIGS. 34A-34F illustrate an LAA low density center ePTFE encapsulation seal device for use with the atrial appendage application in accordance with another embodiment;

FIG. 35 is an ePTFE encapsulation seal for use in vertebral plasty application in accordance with one embodiment;

FIG. 36 illustrates a bone with cavity in vertebral body reconstruction application of the ePTFE encapsulation seal of FIG. 35 in accordance with one embodiment;

FIG. 37 illustrates the introduction of a guide catheter through the bone to the cavity for vertebral plasty application of the ePTFE encapsulation seal of FIG. 35 in accordance with one embodiment;

FIG. 38 illustrates the ePTFE encapsulation seal of FIG. 35 introduced into the bone cavity via the guide catheter in accordance with one embodiment;

FIG. 39 illustrates the expansion of the ePTFE encapsulation seal of FIG. 35 with the introduction of the fluid therein through the injection port via the guide catheter in accordance with one embodiment;

FIG. 40 illustrates the expansion of the bone by the inflated ePTFE encapsulation seal of FIG. 35 in accordance with one embodiment;

FIG. 41 illustrates the further expansion of the bone by the inflated ePTFE encapsulation seal of FIG. 35 in accordance with one embodiment;

FIG. 42 illustrates fully expanded ePTFE encapsulation seal of FIG. 35 in the bone cavity and the guide catheter removed in accordance with one embodiment;

FIG. 43 illustrates a magnified view of the guide catheter showing a separate injection port of the ePTFE encapsulation seal of FIG. 35 as applied in vertebral plasty application in accordance with one embodiment;

FIG. 44 illustrates ePTFE encapsulation seal for vertebral plasty double balloon in accordance with one embodiment;

FIG. 45 illustrates the ePTFE encapsulation seal of FIG. 44 as applied to spinal discs in accordance with one embodiment;

FIG. 46 illustrates a flat NiTi sheet for use as a stent in accordance with one embodiment;

FIG. 47 illustrates the NiTi sheet of FIG. 46 cut or etched in accordance with one embodiment;

FIG. 48 illustrates the cut or etched NiTi sheet of FIG. 47 rolled into a tube in accordance with one embodiment;

FIG. 49 illustrates the rolled NiTi sheet of FIG. 48 covered with ePTFE in accordance with one embodiment;

FIG. 50 illustrates a NiTi sheet folded (or rolled) for delivery in accordance with one embodiment;

FIG. 51 illustrates a NiTi ribbon shaped into a helix for use as a support member in a tubular encapsulation in accordance with one embodiment;

FIG. 52 illustrates a single lumen injection tube with a cutting member for use with the encapsulation device in accordance with one embodiment;

FIG. 53A illustrates the injection tube of FIG. 52 with the cutting member to engage with the encapsulation device in accordance with one embodiment;

FIG. 53B illustrates the injection tube of FIG. 52 after the cutting member has engaged with the encapsulation device in accordance with one embodiment;

FIGS. 54A-54B illustrates encapsulation filler valve male and female components, respectively, in accordance with one embodiment;

FIGS. 55A-55C illustrate the operation of an injection port valve for use with the encapsulation device in accordance with another embodiment;

FIG. 56 is a three-dimensional perspective view of the encapsulation device in accordance with another embodiment of the present invention;

FIG. 57 illustrates a dual layer encapsulation device in accordance with one embodiment of the present invention;

FIGS. 58A-58C illustrate the process of lumbar vertebrae degeneration commonly referred to as herniated disk;

FIG. 59 illustrates the encapsulation device as applied to treat ruptured spinal disks in accordance with one embodiment; and

FIG. 60 illustrates the encapsulation device as applied to treat ruptured spinal disks in accordance with another embodiment.

### DETAILED DESCRIPTION OF THE INVENTION

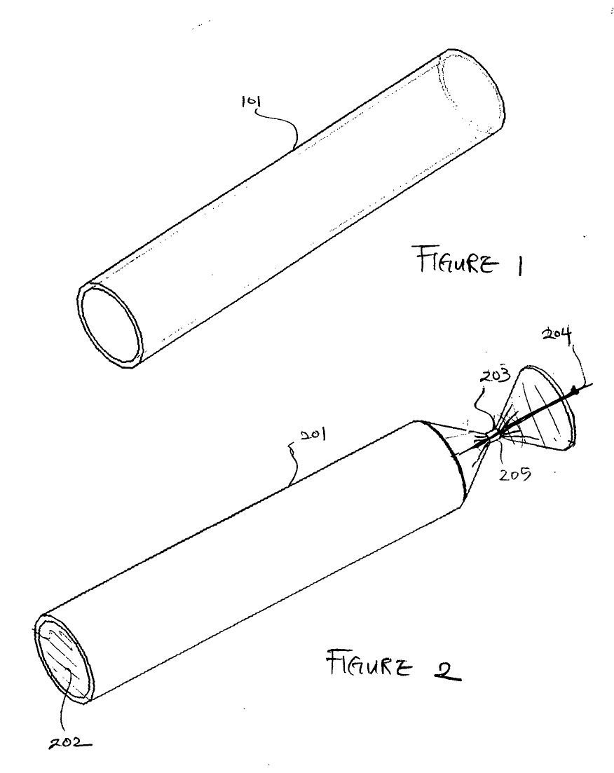

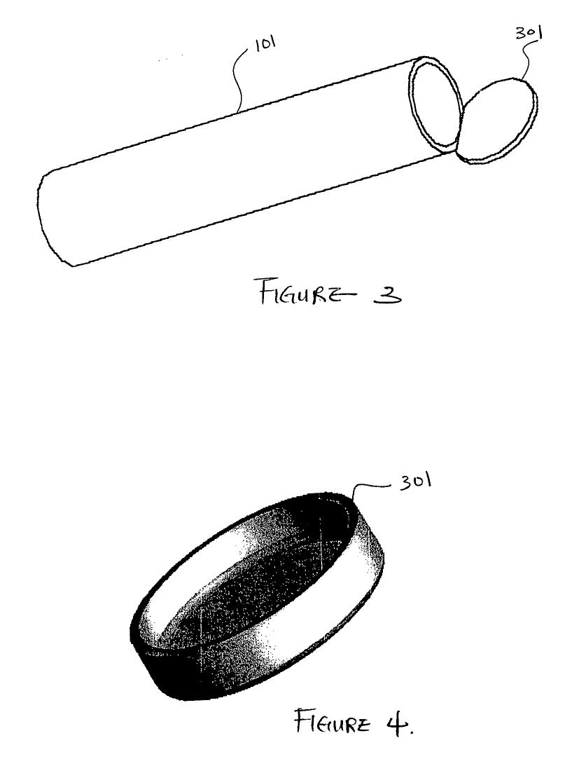

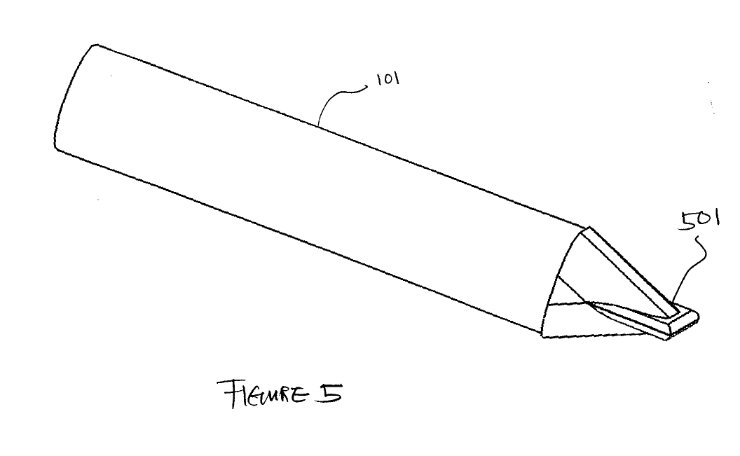

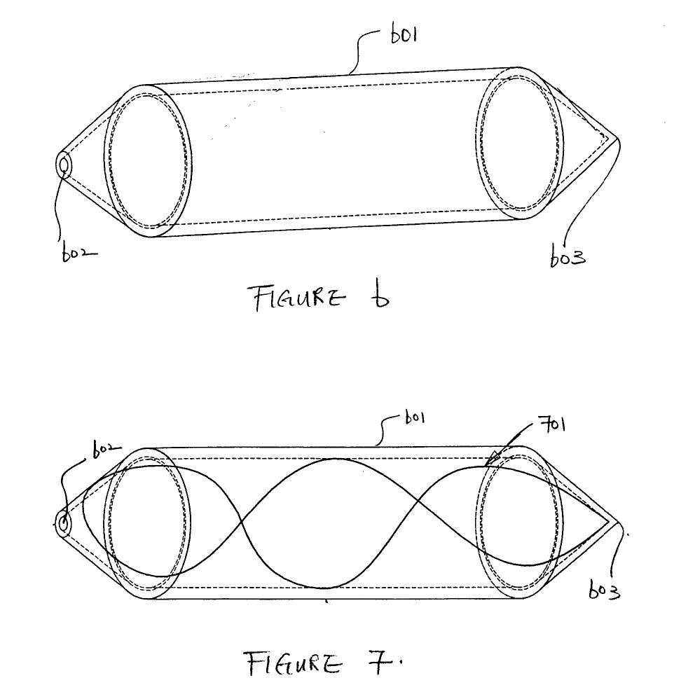

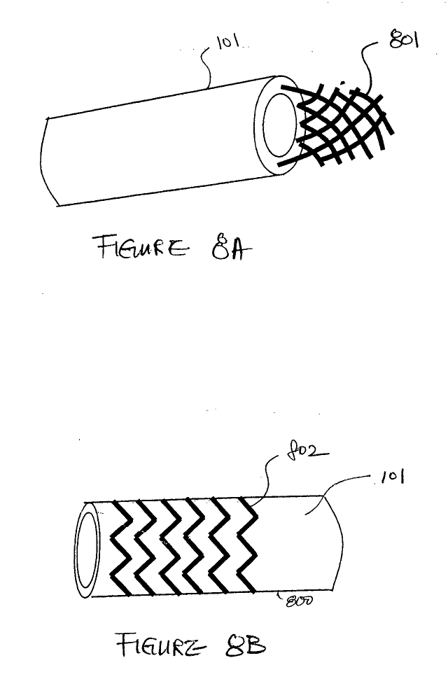

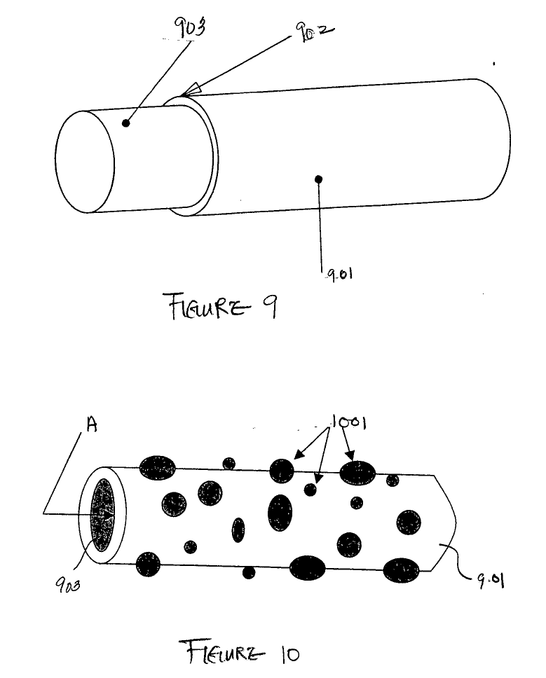

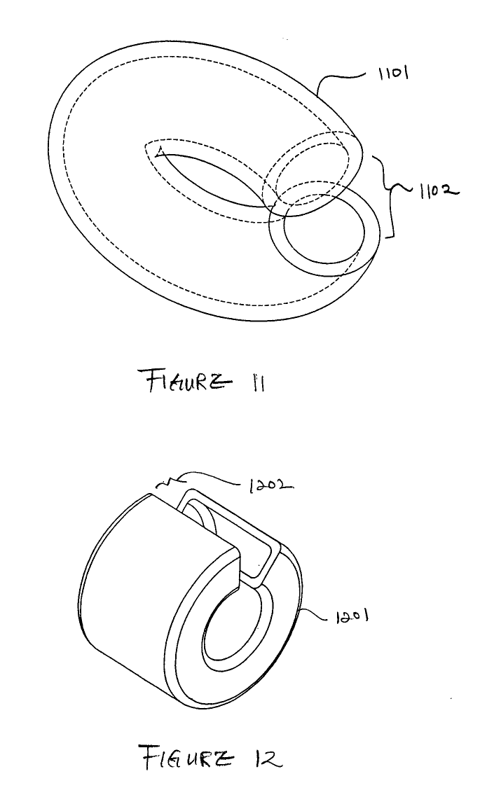

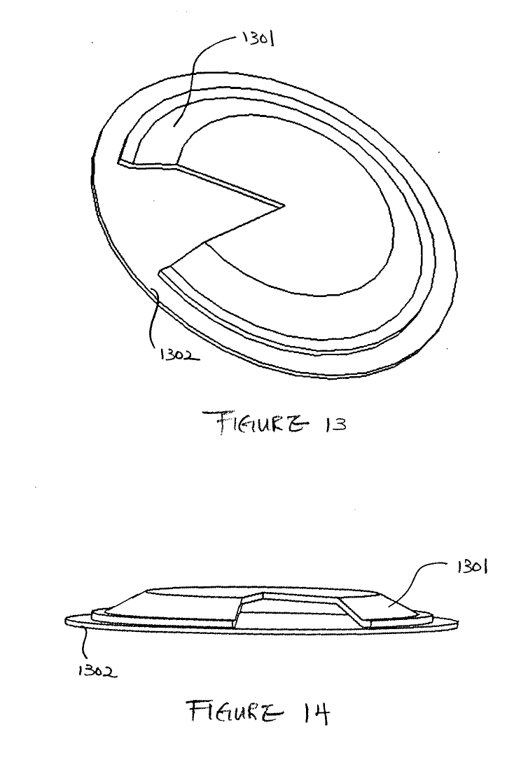

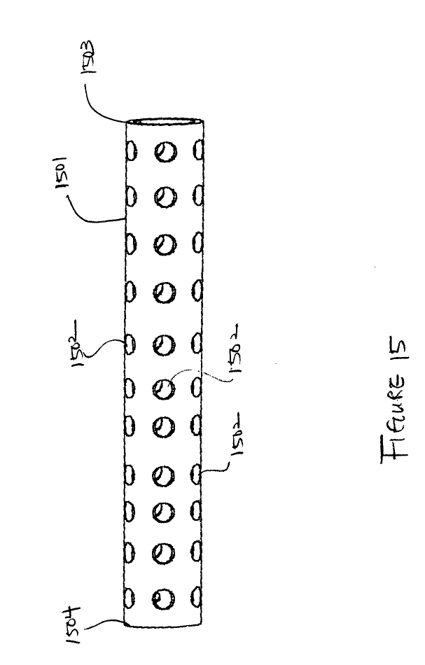

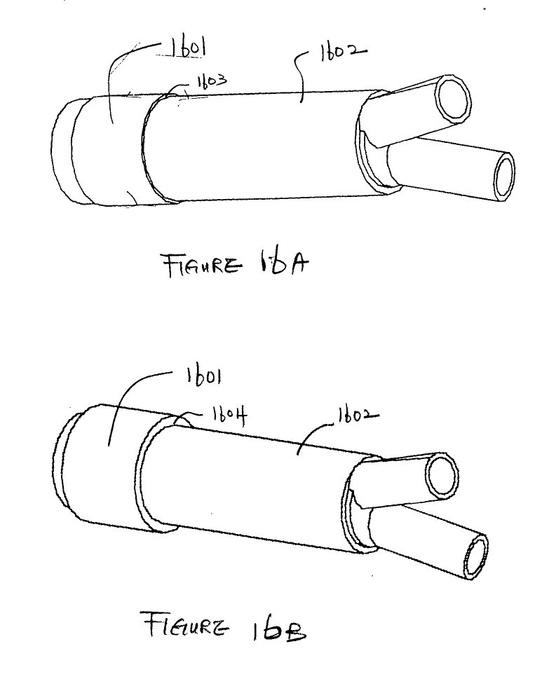

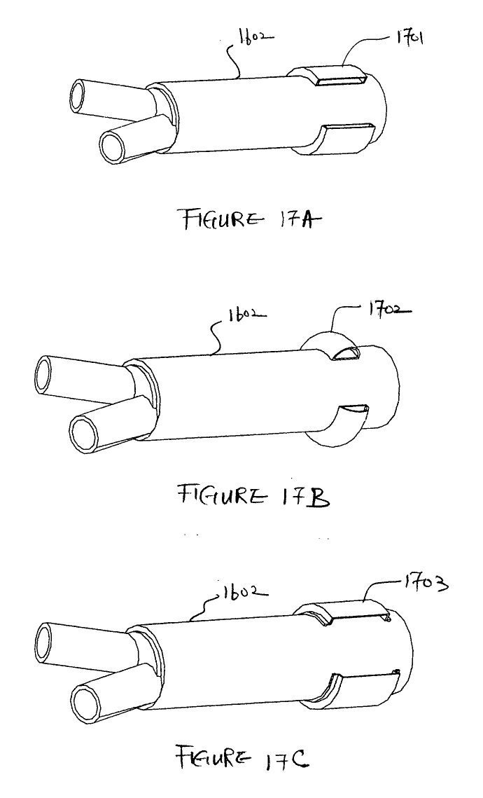

FIG. 1 is a three-dimensional perspective view of a membrane for use as an encapsulation device in one embodiment. Referring to FIG. 1, encapsulation membrane 101 is shown in tubular form. In one embodiment, the encapsulation device may be comprised of membrane 101 which includes expanded Polytetrafluoroethylene (ePTFE) that is made by expanding PTFE tubing under controlled conditions during the manufacturing process. The amount of expansion in ePTFE during manufacturing process is typically referred to as an internodal distance that typically ranges between 1 micron to 200 microns. The manufacturing process alters the physical properties of the PTFE tubing by creating microscopic pores in the structure of the PTFE tubing.

In this manner, the ePTFE differs from the conventional PTFE tubing in that the ePTFE material is microporous, soft, flexible, has a lower dielectric constant, increased linear strength, and improved biocompatibility. The structure of ePTFE is unique in that the material is made up of a number of solid nodes inter-connected by a matrix of thin fibrils. The gaps, or pores between the fibrils permits compatibility with applications that require cellular ingrowth. More specifically, ePTFE's endothelization and thrombogenic properties can be suitably applied for appropriate medical treatments.

In a further aspect of the present invention, the membrane used for the encapsulation device may include porous Polyethylene Terephthalate (PET). More specifically, an ultra-thin walled PET balloon may be used to create a microporous membrane with hole sizes ranging from submicron to a several microns in diameter. In a single PET balloon, hundreds of holes may be placed thereon, providing an improved method of delivering the desired fluid because the pore size may be controlled precisely, allowing very small amounts of the intended fluid to be infused over a well-defined and desired area.

Moreover, in another embodiment of the present invention, the ePTFE encapsulation device may be provided with a PET backbone to provide additional structural support. As discussed in further detail below, a porous PET shell may be provided around the outside surface of the ePTFE encapsulation device to further strengthen and reinforce the structural integrity of the encapsulation device. Within the scope of the present invention, the porous shell around the outside surface of the ePTFE encapsulation device may also include a metallic tube.

Additionally, while ePTFE and porous PET are discussed above as membrane material for use as the encapsulation device, other porous membrane material may be used within the scope of the present invention such as metallic wire mesh or porous Nitinol. More specifically, in one embodiment, the encapsulation device of the present invention may be made from material that is semi-permeable on one side and relatively less permeable or non-permeable on the other side. The more permeable side could be designed to weep some of the more viscous material, such as an adhesive including cyanoacrylate, epoxy, or other bioactive compound, into and/or through the encapsulation device membrane, to preferentially activate or adhere to different areas along the geometry of the encapsulation device. The membrane itself may be dumbbell shaped and have the cavity filled or layered, with channels between the layers filled.

FIG. 2 is a three-dimensional perspective view of one embodiment of an encapsulation device using the membrane 101 shown in FIG. 1. Referring to FIG. 2, the encapsulation device 201 has a sealed end 202 and a self-sealing end 203. The self-sealing end 203 of the encapsulation device 201 may be configured to include a valve mechanism. In one embodiment, the self-sealing end 205 is configured to receive an injection port 204 of a catheter (not shown) discussed in further detail below, to permit the introduction of the desired fluid into the encapsulation device 201.

As can be seen, the encapsulation device 201 of one embodiment includes a hollow, sealed porous configuration with the valve mechanism 205 for introducing one or more fluids therein. For example, a first fluid, such as saline solution, may be introduced through the valve mechanism 205 to inflate the encapsulation device 201 to ensure it was placed properly. The first fluid then may be displaced with a second fluid, such as an adhesive, to secure the encapsulation device 201 in place, whereby the first fluid weeps through the walls of the encapsulation device 201, while the secondary fluid may partially weep therethrough. In a further embodiment, upon completing the fluid weeping or displacement, the valve mechanism 205 may be sealed completely.

In one embodiment, an injection port 204 of a catheter tube (not shown) may be placed inside encapsulation device 201 via the valve mechanism 205 thus creating a filler tube. In such embodiment, when the first fluid in the encapsulation device 201 is displaced with the second fluid, the injection port 204 may be removed, and the valve mechanism 205 sealed.

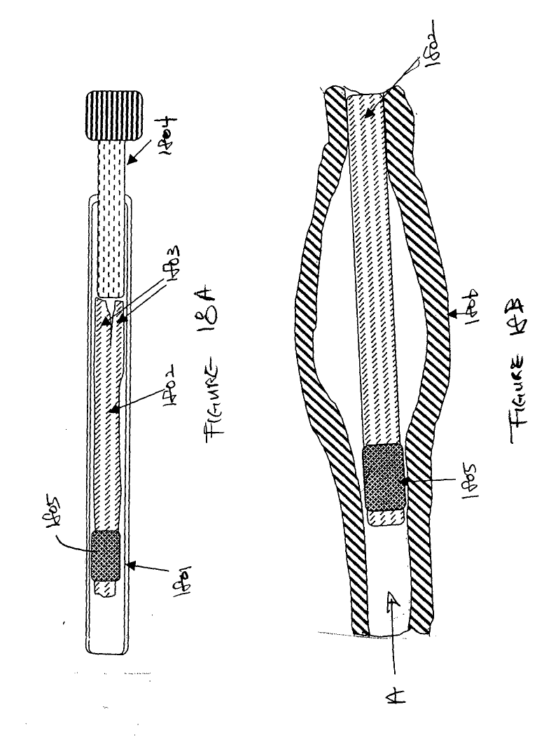

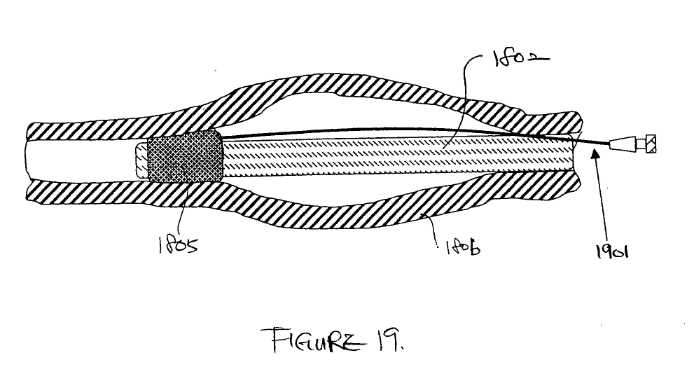

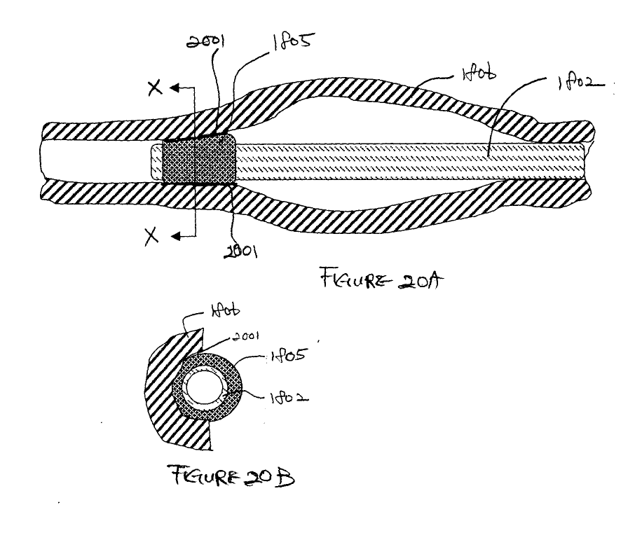

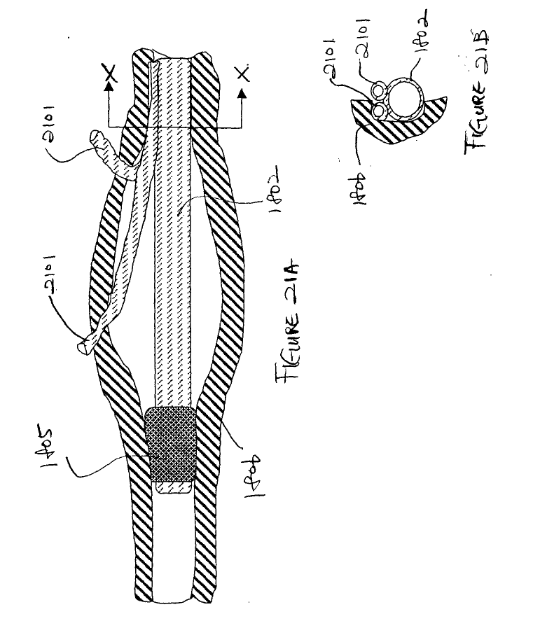

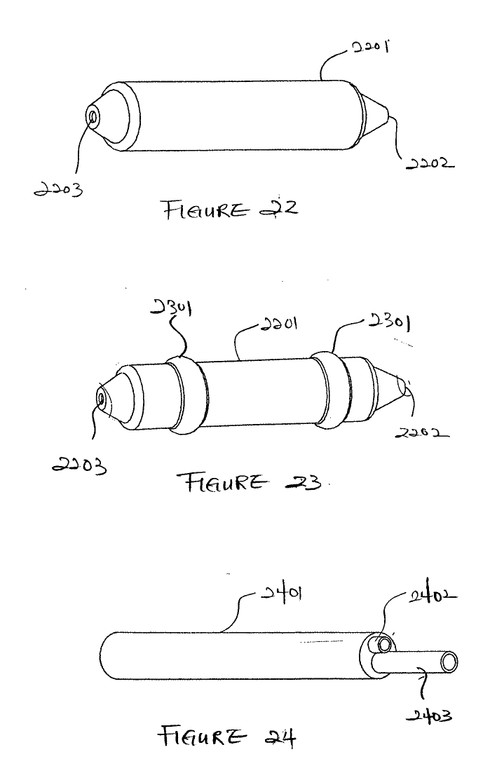

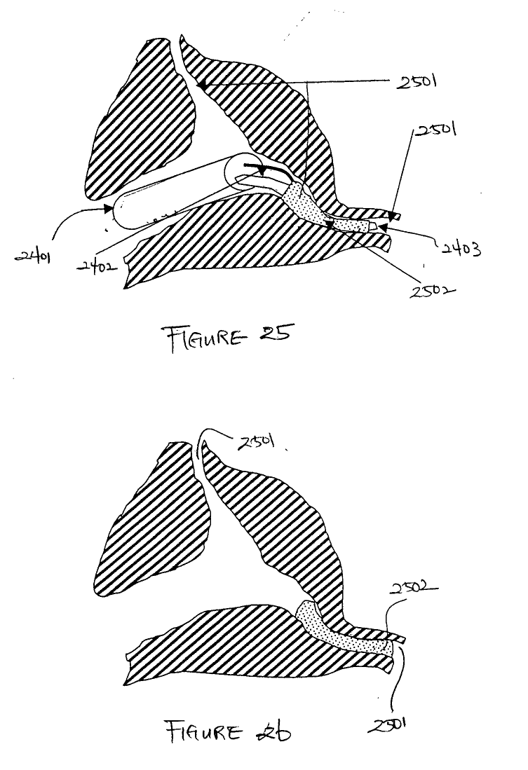

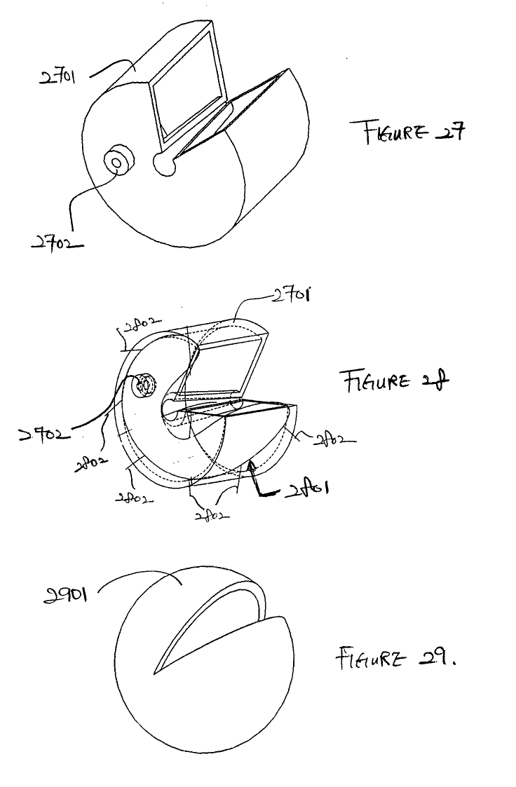

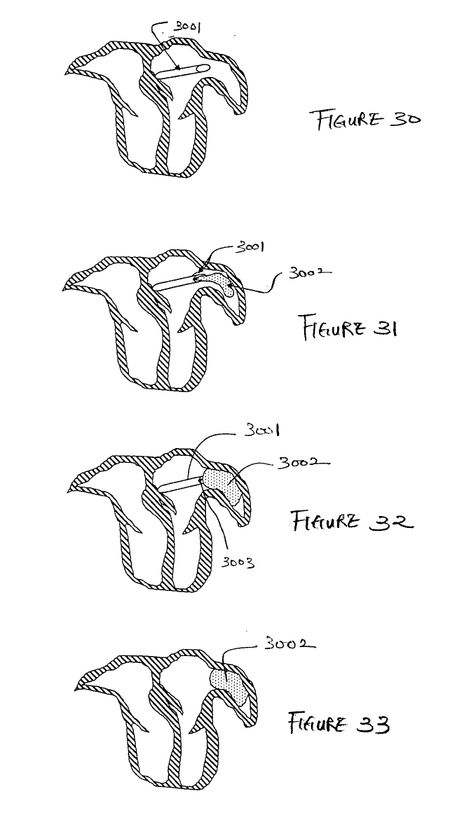

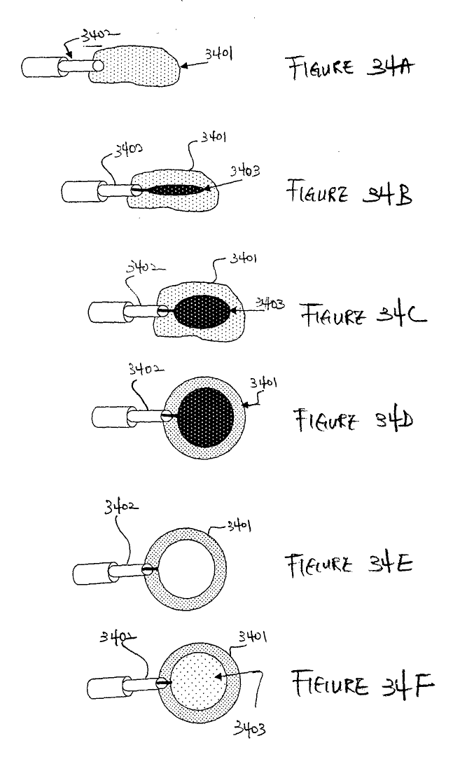

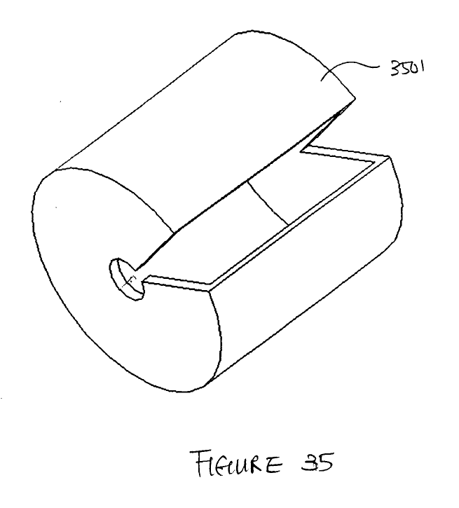

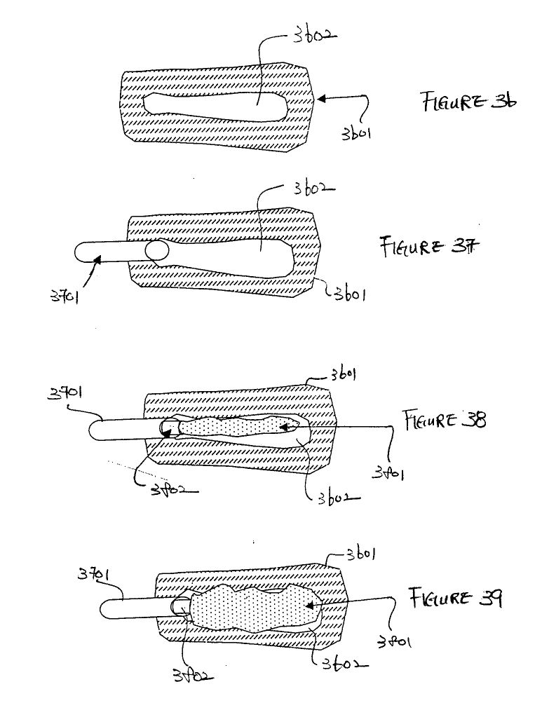

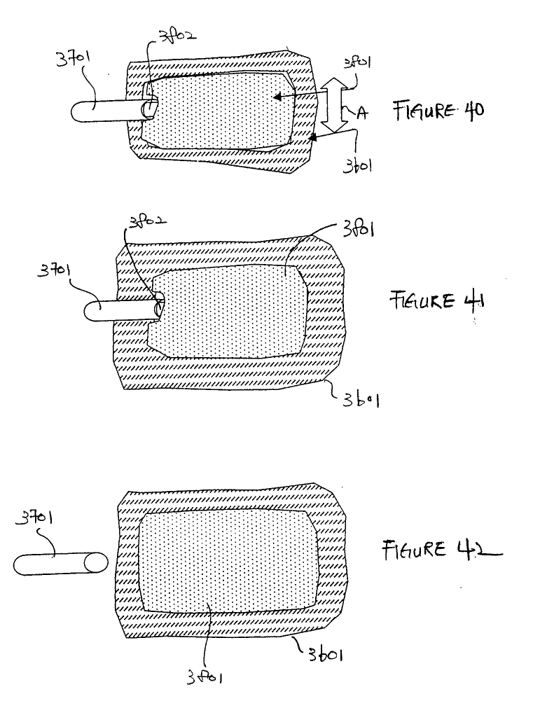

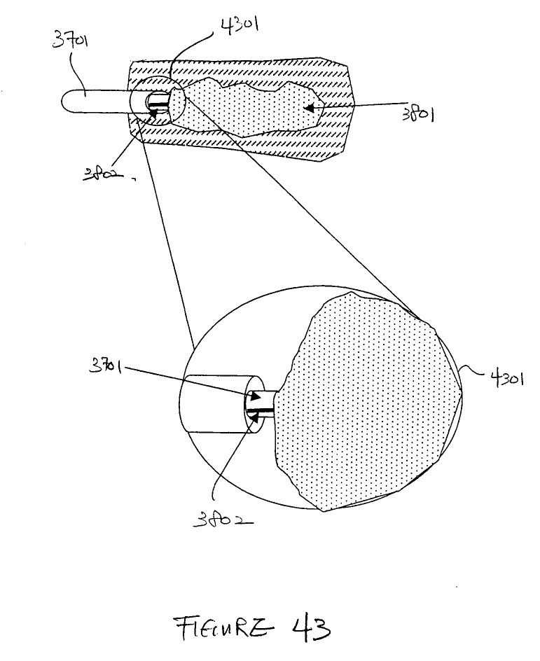

The sealed end 202 of the encapsulation device 201 may be formed in various ways. For example, an end cap portion 301 may be substantially severed from the structure wall of the encapsulation membrane 101 (FIG. 1), as illustrated in the three-dimensional perspective view in FIG. 3. Alternatively, an end cap portion 301 may be completely severed from the encapsulation membrane 101 (FIG. 1), as illustrated in the three-dimensional perspective view in FIG. 4. The end cap 301 is then bonded to the end of the encapsulation membrane 101 (FIG. 1) to form the sealed end 202 of the encapsulation device 201 (FIG. 2) using bonding techniques discussed below. In yet a further aspect of the present invention, the sealed end 202 of the encapsulation device 201 may be formed by cutting part of the wall of the encapsulation membrane 101 (FIG. 1) and folding it over the end section and bonding it.

In still yet another embodiment, the sealed end 202 of the encapsulation device 201 may be formed by pinching an end of the encapsulation membrane 101 (FIG. 1) to form a seal, as illustrated in the three-dimensional perspective view of the encapsulation device 201 in FIG. 5.

In one embodiment, the encapsulation device 201 may include sealed ends 202, 203 (FIG. 2) which may be achieved by applying an adhesive such as, for example, cyanoacrylate, to the inner diameter of the encapsulation membrane 101 (FIG. 1) at the end portion of the membrane 101, and then by twisting the an portion until the adhesive cures. This is illustrated in FIG. 2 by the twisted self-sealing end 203 of the encapsulation device 201. Alternatively, a band (not shown) could be slid or crimped over the self-sealing end 203 (FIG. 2) and the adhesive could be bonded to the inside of the band as well as to the outside of the self-sealing end 203, or a combination of the two. Thereafter, the excess membrane end portion may be trimmed off and discarded.

FIG. 6 is a three-dimensional perspective view of the encapsulation device in accordance with yet another embodiment. Referring to FIG. 6, encapsulation device 601 comprises an ePTFE membrane and includes a self-sealing end 602 and a sealed end 603. In one embodiment, the wall thickness of the ePTFE encapsulation device 601 may range approximately between 0.004 inches to 0.025 inches, and the internodal distance may range approximately between 1 to 200 microns.

FIG. 7 is a three-dimensional perspective view of the ePTFE encapsulation device 601 of FIG. 6 in accordance with another embodiment. As shown, there is also provided a wire reinforcement 701 within the encapsulation device 601 which is configured to assist the encapsulation device 601 maintain its shape during delivery, deployment and hydrating of the encapsulation device 601. In one embodiment, the wire reinforcement 701 may include Nickel Titanium alloy (NiTi), stainless steel (such as 304V, 302 or 316), and/or some structural polymer. In one embodiment, the diameter of the wire reinforcement may be approximately 0.014 inches. The wire reinforcement 701 may include Super Elastic Nitinol (SEA NiTi) to allow it to be heat shaped prior to placement in the encapsulation device. The wire reinforcement 701 may include stress (or temperature) induced Martensite to allow it to be collapsible until it is delivered to a desired location. The wire reinforcement 701 may include stainless steel which may be bent or manipulated to a desired shape. In one embodiment, the wire reinforcement 701 ranges approximately between 0.005 inches to 0.04 inches in diameter.

The wire reinforcement 701 may permanently remain in the encapsulation device 601, or may be removed prior to, during, or after the encapsulation device 601 is properly positioned. A removable wire reinforcement 701 may need to have PTFE or a hydrophyllic coating on it to allow it to be easily removed. The wire reinforcement 701 may be bonded to the membrane of the encapsulation seal, or alternatively, may freely float within the seal.

In one embodiment, the wire reinforcement 701 may be secured to the wall of the encapsulation device 601 or loosely provided within the encapsulation device 601. Furthermore, in accordance with one embodiment of the present invention, the wire reinforcement 701 may be straight, or have tapered or stepped grinds to help obtain desired shape of the encapsulation device 601.

FIGS. 8A-8B are three-dimensional perspective views of the ePTFE encapsulation device in accordance with still another embodiment of the present invention. In such embodiment, NiTi wire reinforcement 801 as shown in FIG. 8A is woven and then attached to an outer surface of encapsulation membrane 101 to form the main body of the encapsulation device 800, as shown in FIG. 8B. In an alternate embodiment of the present invention, the wire reinforcement 801, 802 may also be provided on the inside of the encapsulation device 201. Furthermore, the wire reinforcement 801, 802 may also be attached with suture or adhesive, or heat bonded and then fused into the encapsulation membrane 101 to form wire reinforced encapsulation device 800. In yet still another embodiment of the present invention, the wire reinforcement 801 may be inserted into the encapsulation device 201 via one of the ends 202, 203 of the encapsulation device 201 (FIG. 2) before the end is sealed. Thereafter, when the end 202, 203 of the encapsulation device 201 is sealed, the wire reinforcement would be too large to pass through the pores of the encapsulation device 201, the sealed end 202, or the self-sealing valve 204 discussed above.

FIG. 9 is a three-dimensional perspective view of one embodiment of an encapsulation device 901 having an open end 902. Referring to the Figure, a displacement fluid 903 is introduced into the open end 902 of the encapsulation device 901. In one embodiment, the displacement fluid 903 may include one of an epoxy, an adhesive, or Super Porous Hydrogel (SPH). In the case where SPH is inside the encapsulation device 901, fluid within the encapsulation device 901 is not displaced, but rather, added to hydrate the SPH. Otherwise, displacement fluid 903 that hardens in one embodiment displaces the saline or other fluid inside the encapsulation device 901 through a weeping (or sweating) process such as, for example, by an exchange through the pores.

FIG. 10 is a three-dimensional perspective view of the encapsulation device 901 illustrating fluid weeping 1001 in accordance with one embodiment of the present invention. Referring to the Figure, arrow A illustrates the directional flow of the displacement fluid 903 (FIG. 9). As a result, fluid already present within the encapsulation device 901 weeps out of the encapsulation device 901 through its pores. More specifically, in one embodiment of the present invention, the introduction of the displacement fluid 903 (FIG. 9), which is a more viscous fluid, into the encapsulation device 901 pushes out the existing fluid, such as saline, present within the encapsulation device 901. Alternatively, in another embodiment, the fluid in the encapsulation device 901 may be exchanged back through the delivery catheter (not shown).

Due to the viscosity of the displacement fluid 903, it does not weep as readily as the prior fluid present in the encapsulation device 901, if at all. The displacement fluid 903 then hardens or cures within the inner space of the encapsulation device 901, allowing the encapsulation device 901 to take the final shape of the form of the intended target location. In one embodiment, the displacement fluid 903 may be an adhesive that bonds the encapsulation device 901 in its desired place during application. This may be achieved by a slight weeping of the displacement fluid 903 through the pores of the encapsulation device 901 after having displaced the first fluid that was in the encapsulation device 901.

FIG. 11 is a three-dimensional perspective view the encapsulation device in accordance with still another embodiment of the present invention. Referring to FIG. 11, the encapsulation device 1101 is configured in a circular tube shape. A section 1102 of the encapsulation device 1101 is cut away for purposes of illustrating the inner cavity of the encapsulation device 1101. In one aspect of the present invention, the encapsulation device 1101 shown in FIG. 11 may be used as an encapsulation seal for the treatment of Abdominal Aortic Aneurysm (AAA). In such embodiment, the encapsulation device 1101 may be adhesively bonded or attached with suture to an AAA stent. In one embodiment, and the encapsulation device 1101 may also include wire reinforcement within the scope of the present invention.

FIG. 12 is a three-dimensional perspective view of the encapsulation device in accordance with yet still another embodiment of the present invention. As shown in FIG. 12, the encapsulation device 1201 is configured in a rectangular tube shape with a substantially corresponding cavity therein. As with the encapsulation device 1101 shown in FIG. 11, the cut away section 1202 shown in FIG. 12 is solely for purposes of illustrating the inner cavity of the encapsulation device 1201.

FIG. 13 is a three-dimensional perspective view of the encapsulation device in accordance with a further embodiment of the present invention, and FIG. 14 is a side view of the ePTFE encapsulation device shown in FIG. 13. As shown in FIGS. 13-14, there are provided two encapsulation membranes 1301, 1302 which are bonded to each other to form the encapsulation device. In this embodiment, the two encapsulation membranes 1301, 1302 are bonded together with an adhesive, or alternatively, heat bonded together. In the case where the membranes 1301, 1302 are heat bonded together, a thin layer of fluorinated ethylene propylene (FEP) may be provided between the membrane layers that will melt into the pores of the encapsulation 1302, 1302 membrane and complete the seal. Alternatively, the encapsulation membranes 1301, 1302 may be cut into a circular pattern to form disc shapes and folded over and/or bonded. In one embodiment, the heat bonding may be achieved at a temperature of approximately 650 to 700 degrees Fahrenheit for a period of approximately 4 to 60 seconds. In yet another embodiment, the bonding may be achieved by a combination of heat and applied pressure using, for example, two hot plates which are configured to clamp down at the desired bonding location. In this case, higher applied pressure requires less heat, and likewise, lower applied pressure requires higher heat to obtain substantially the same bonding result. The wall thickness of the encapsulation membrane may range approximately between 0.002 inches and 0.05 inches. The ePTFE may also be bonded using other standard ePTFE bonding techniques such as ultrasonic welding.

FIG. 15 illustrates an encapsulation device in accordance with yet another embodiment of the present invention. Referring to FIG. 15, the encapsulation device 1501 comprises a nitinol tube whose tubular wall is made porous with laser drilled holes 1502 therein. In one embodiment, the size of the laser drilled holes 1502 may range in approximately 0.001 inches to 0.05 inches in diameter, while the thickness of the nitinol tube encapsulation device 1501 may range approximately between 0.002 inches to 0.02 inches. Furthermore, as with the previously discussed embodiments of the encapsulation device, in application, the end portions 1503, 1504 of the nitinol tube encapsulation device 1501 are sealed. Moreover, if the laser drilled holes 1502 are relatively large, the adhesive for use as displacement fluid is desired to be substantially thick so as to have gel-like constituency.

Indeed, within the scope of the present invention, the encapsulation membrane is not limited to polymetric materials, but rather, also includes a metal tube, either thin walled flexible or thick and stiff. In this aspect of the present invention, the metal tube may include tiny holes laser drilled into its wall. It should be appreciated by those skilled in the art that there are other methods of making Nitinol porous. There could also be a pattern laser cut to make the metal tube flexible. Examples of metals used in the metal tube may include, for example, Nickel Titanium alloy (NiTi), or stainless steel (such as types 304, 302, and 316).

In the manner described above, in accordance with the various embodiments of the present invention, a broad platform is provided for an encapsulation device in a three-dimensional configuration by using a porous membrane to create the outer surface membrane of the three-dimensional shape. In application, the three-dimensional shape such as a dumbbell shape may be actualized or deployed by filling it with saline to determine if the size and location are optimal. When the final location and size are acceptable, the fluid within the encapsulation device may be displaced through the permeable membrane with a more viscous material, that would not readily pass through the membrane and may change state or harden due to the membrane pore size. In one aspect, the Super Porous Hydrogel (SPH) may be used to fill the encapsulation device such that the encapsulation device swells to take the shape of the restrictive encapsulation membrane. The SPH material does not pass through any pores in the membrane of the encapsulation device, but rather, is contained within the encapsulation device and forms the desired shape.

Moreover, either in the case of thoracic aortic aneurysm (TAA) or abdominal aortic aneurysm (AAA), the encapsulation device in accordance with the various embodiments of the present invention may be deployed on a temporary basis at the desired location in the patient prior to transporting the patient to a hospital for surgery to treat the condition. This is important especially in circumstances where the aneurysm, without a temporary relief, may be fatal to the patient even before the patient can be transported to the hospital. In this embodiment, the encapsulation device may be directly filled with the desired fluid rather than performing a displacement process weeping out a first fluid and then introducing a second fluid which displaces the first fluid within the encapsulation device. Indeed, the encapsulation device may be quickly and easily positioned at the aneurysm location for providing temporary relief, and during surgical operation, the encapsulation device may be removed and replaced with a permanent device.

Conventional abdominal aortic aneurysm (AAA) stents have been known to develop leaks at the ends of the stent graft or prosthesis referred to as endoleaks. The endoleaks can allow blood to flow into the aneurysm, negating the effect of the endovascularly placed prosthesis, and potentially resulting in fatal consequences. As discussed in further detail below in conjunction with FIGS. 16-21, encapsulation seals in accordance with various embodiments of the present invention may be provided on the AAA prosthesis so as to substantially prevent the potential for endoleaks.

FIGS. 16A-16B illustrate an encapsulation seal 1601 uninflated and inflated, respectively, in accordance with one embodiment of the present invention. More specifically, the encapsulation seal 1601 shown in FIGS. 16A-16B is configured for use in treating abdominal aortic aneurysm, and is shown as bonded to a prosthesis (stent) 1602 in the Figure. As shown by the thickness 1603 of the encapsulation seal 1601 in FIG. 16A compared with the thickness 1604 of the encapsulation seal 1601 shown in FIG. 16B, the encapsulation seal 1601 in one embodiment is deployed in the deflated state shown in FIG. 16A to the target area for treatment with the prosthesis 1602. Thereafter, the encapsulation seal 1601 is inflated as shown in FIG. 16B, once it is determined that the encapsulation seal 1601 is properly positioned at the desired location. In one aspect, the encapsulation seal 1601 may include wire reinforcement as discussed above.

FIGS. 17A-17C illustrate the encapsulation seals formed by a flat rectangular tubular membrane, a round tubular membrane, and a bonded ePTFE membrane, respectively, which is bonded to the prosthesis (stent) 1602 in accordance with one embodiment. As discussed in conjunction with FIGS. 13-14, the ePTFE membrane forming the encapsulation seal 1703 of FIG. 17C is bonded along the edges. It will be appreciated that the cut away portion of the encapsulation seals 1701, 1702, and 1703 shown in each of FIGS. 17A-17C, respectively, are solely for purposes of illustrating the inner cavity thereof, and the encapsulation seals 1701-1703 may be configured to wrap around the entire circumference of the prosthesis 1602.

FIG. 18A illustrates a prosthesis 1802 with an encapsulation seal 1805 for use as an AAA device in accordance with one embodiment of the present invention. Referring to the Figure, there is shown a catheter tube 1801 made of polyethelyne, a prosthesis 1802 in collapsed state showing the legs 1803 of the prosthesis 1802, and a prosthesis push rod 1804. At one end of the prosthesis 1802, opposite the legs 1803, is shown an encapsulation seal 1805. The prosthesis 1802 is configured to be collapsible and may be delivered using a 16 French delivery catheter, for example. It should be noted that the delivery catheter for use to deliver the prosthesis 1802 within the scope of the present invention may include other sizes as appropriate for the desired application. The prosthesis 1802 may comprise of Nickel Titanium alloy (NiTi) and a graft material, potentially ePTFE, in one embodiment.

FIG. 18B illustrates the prosthesis 1802 of FIG. 18A inside an aortic aneurysm in accordance with one embodiment of the present invention. Referring to FIG. 18B, the flow of blood is in the direction shown by arrow A within the vessel wall 1806. While not shown in FIG. 18, the legs 1803 (FIG. 18AB) of the prosthesis 1802 are configured to channel blood flow to the left and right legs.

FIG. 19 illustrates the prosthesis of FIG. 18A inside the aortic aneurysm with a catheter 1901 attached to the encapsulation seal 1805, for example via a valve mechanism (not shown), in accordance with one embodiment of the present invention. In one embodiment, the catheter 1901 may include a small single lumen catheter. Referring to the Figure, the encapsulation seal 1805 may be filled with fluid via catheter 1901. In particular, in one embodiment, the encapsulation seal 1805 is first filled with saline solution to assist the encapsulation seal 1805 in reaching the target location. Thereafter, when it is determined that the encapsulation seal 1805 is properly positioned at the target location, a displacement fluid is introduced into the encapsulation seal via catheter 1901.

When the encapsulation seal 1805 is filled with the desired fluid via the catheter 1901, the catheter 1901 is retracted and removed from the blood vessel. As discussed above, the displacement fluid has a higher viscosity than the saline solution, but could still slightly flow through the pores of the encapsulation seal 1805. The saline solution weeps out of the pores of the encapsulation seal 1805, and thereafter, the displacement fluid is cured such that the encapsulation seal 1805 is bonded to the vessel wall 1901 at the target location.

FIG. 20A illustrates the prosthesis of FIG. 18A with the encapsulation seal 1805 filled and in place at target location 2001 of the vessel wall 1806, in accordance with one embodiment of the present invention. And, FIG. 20B illustrates a cross-sectional view of the prosthesis along the line X shown in FIG. 20A in accordance with one embodiment. As shown in the Figures, once cured, the encapsulation seal 1805 is bonded to the vessel wall 1806 at the target location 2001, to help prevent endoleaks at the end of the prosthesis 1802.

In the manner described above, in accordance with the various embodiments of the present invention, an encapsulation seal 1805 may be provided to the main body of the prosthesis 1802 to prevent endoleaks. In one embodiment, an encapsulation seal 1805 in the shape of a donut or a cylinder is provided around the outside of the main body of the prosthesis 1802. In one aspect, the wall thickness of the encapsulation seal 1805 may range approximately from 0.004 inches to 0.02 inches. Furthermore, as discussed above, within the scope of the present invention, the encapsulation seal 1805 may have different shapes, such as a flat outside and inside, potentially making the encapsulation seal 1805 longer longitudinally. Alternatively, the encapsulation seal 1805 may comprise one single sheet of the ePTFE membrane bonded along the edges of the sheet to the main body of the prosthesis 1802, thus forming a donut shape with the inside of the donut shape being the outer wall of the prosthesis 1802. This bond along the edge of the ePTFE membrane may, in one embodiment, be a few millimeters wide. Further, the porosity of the encapsulation seal 1805 may be greater than the prosthesis graft so that the displacement fluid weeps out of the encapsulation seal 1805 rather than into the prosthesis 1802.

Yet another embodiment of the encapsulation seal 1805 may include a twisted end overlapping where the other end of the encapsulation seal 1805 is around the main body of the prosthesis 1802. Alternatively, the encapsulation seal 1805 may include a capped end portion using, for example, end cap portion 301 (FIG. 3). The end cap portion, formed from a cut in the wall of the encapsulation membrane or a completely separate piece of the encapsulation membrane, is bonded or molded to the encapsulation seal 1805.

Still a further embodiment of the present invention may include the use of a wire reinforcement (for example, as discussed above in conjunction with FIGS. 7-8) for the encapsulation seal 1805 to assist in forming a tight closure against the vessel wall 1806. For example, the wire reinforced encapsulation seal 1805 may provide support during the delivery of encapsulation seal 1805 to the target location or help the encapsulation seal 1805 take the correct shape upon deployment at the target location. The wire reinforcement may comprise stainless steel, nickel titanium (NiTi), or a suitable structural polymer. In the case of Super Elastic Nitinol (SEA NiTi) as the wire reinforcement, it could be heat shaped.

FIG. 21A illustrates a prosthesis with an encapsulation seal for use as an AAA device in an aneurysm with side branch arteries in accordance with one embodiment, and FIG. 21B illustrates cross-sectional view of the prosthesis with the encapsulation seal along the line X shown in FIG. 21A. As shown in the Figures, there are circumstances where side branch arteries 2101 are found in an aneurysm that can feed blood flow into the aneurysm potentially adversely impacting the use of the AAA device (i.e., to prevent blood flow into the aneurysm). Accordingly, in one embodiment of the present invention, the position of the prosthesis 1802 within the vessel wall 1806 effectively pinches the side branch arteries 2101 to restrict the blood flow therethrough into the aneurysm. Furthermore, the area between the outer wall of the prosthesis 1802 and the inner surface of the vessel wall 1806 and surrounding the side branch arteries 2101 may be filled with adhesive (e.g., UV curable adhesive, for example), hydrogel, or SPH to ensure that blood flow to the aneurysm is avoided.

FIG. 22 illustrates an encapsulation device 2201 in accordance with yet another embodiment of the present invention. As shown in the Figure, the encapsulation device 2201 is configured to be used as a contraceptive device using an ePTFE membrane with a sealed end 2202 and a self-sealing valve 2203 at the opposite end. In one embodiment, the self-sealing valve 2203 is sealed after the catheter (not shown) is pulled out, while the sealed end 2202 is sealed at the time of manufacture. In one aspect, the encapsulation device 2201 may be filled with SPH, adhesive or any other suitable fluid.

FIG. 23 illustrates the encapsulation device of FIG. 22 in accordance with another embodiment of the present invention. As shown in the Figure, the encapsulation seal 2201 is provided with a plurality of ribs 2301 which are configured to assist in mechanically locking the encapsulation device after the device is deployed to the desired location and detached from the guide catheter and the like. For example, in one embodiment, in the case where the encapsulation device is in a simple tubular shape, there is a potential for the device to migrate from the initially deployed location. While the adhesive weeped though the pores of the encapsulation device is configured to bond it in its place, the plurality of ribs 2301 may be configured as a mechanical lock as they are larger in diameter than the body of the encapsulation device. Typically, the size of the encapsulation device for a given application is over-estimated by a predetermined percentage (for example, 10%) of the diameter of the encapsulation device. The ribs 2301 are then designed to protrude an additional amount (such as, for example, 10%) helping to keep the device from moving from the deployed location, while avoiding any significant strain on the vessel or lumen in which the device is deployed.

Additionally, the plurality of ribs 2301 may help to prevent fluid from passing by the encapsulation device. That is, as arteries and other parts of the human body in which the encapsulation device is deployed is not shaped completely circular, but rather, take the shape of an oblong or an oval, for example, the ribs 2301 may assist the deployed encapsulation device stop any fluid passing by the body of the device if the artery (or other lumen) is not completely round.

FIG. 24 illustrates a two-lumen contraceptive catheter for use with the encapsulation device 2201 shown in FIG. 22 in accordance with one embodiment of the present invention. Referring to FIG. 24, the two-lumen contraceptive catheter 2401 includes a short lumen 2402 configured as the inflation lumen, and a long lumen 2403 configured as the guidewire lumen. In this configuration, the encapsulation seal (not shown) is configured to operatively couple to the longer guidewire lumen 2403 when deployed to the target location. In one embodiment, the main body of the catheter 2401 may comprise of an extruded polymer such as 63D PEBAX or nylon, while the short lumen 2402 and the guidewire lumen 2403 may be made of polyimide. In a further embodiment, the guidewire lumen 2403 may be configured to be retractable, and thus may be retracted before inflation of the encapsulation device 2201.

FIG. 25 illustrates an encapsulation device 2502 in application as a contraceptive device in accordance with one embodiment of the present invention. As can be seen from the Figure, the two-lumen contraceptive catheter 2401 (FIG. 24) is inserted into the vagina using a scope (not shown), with the lumen side of the catheter 2401 introduced into the vagina first. The encapsulation device 2502 is coupled to the guidewire lumen 2403 and is initially collapsed for delivery into one of the two fallopian tubes 2501. Thereafter, using the inflation lumen 2402, the implantable encapsulation device 2502 is filled with saline solution. Once it is determined that the encapsulation device 2502 is positioned at the proper target location, a displacement fluid such as an adhesive is provided into the encapsulation device 2502 via the inflation lumen 2402. In this manner, the encapsulation device 2502 may be repeatedly inflated and deflated until it is properly positioned at the target location.

FIG. 26 illustrates the encapsulation device 2502 shown in FIG. 24 in accordance with one embodiment of the present invention after the catheter 2401 is removed. As can be seen from the Figure, the catheter 2401 is removed after the encapsulation device 2502 is properly inflated and attached to the inside of the fallopian tube. It should be noted that one end of the encapsulation device 2502 is sealed prior to the introduction of the displacement fluid, and upon the removal of the catheter 2401, the opposite end of the encapsulation device 2502 must be sealed. In one embodiment, a self-sealing valve may be provided at the opposite end for sealing the end of the encapsulation device 2502 after removing the catheter. Further detailed discussion of the self-sealing valve is provided below.

In the manner described above, in accordance with one embodiment of the present invention, a permanent contraceptive device may be realized. More specifically, an encapsulation device comprising a porous membrane, such as an ePTFE membrane, may be sealed at one end and provided with a self-sealing valve at the opposite end. A physician may inflate the encapsulation device using a first fluid, such as saline solution, properly place the encapsulation device at the desired location (e.g., inside one of the fallopian tubes). Thereafter, the encapsulation device may be filled with an adhesive or hydrogel, via the end having the self-sealing valve, which would harden. The self-sealing valve of the encapsulation device would then be sealed to create the desired blockage of the fallopian tube.

The introduction of the adhesive as the displacement fluid would force the first fluid (e.g., saline solution) to weep through the pores of the encapsulation device, until the adhesive itself is slightly wept through the pores of the encapsulation device to lock the device in place. In one embodiment, an ultra-violet curable adhesive may be used. Optionally, a pusher device (not shown) may be used to hold the encapsulation device in place while the catheter is removed from the self-sealing valve. In another embodiment, the encapsulation device discussed herein may be used in lung reduction for pulmonary procedures.

In accordance with an alternate embodiment of the present invention, the encapsulation device 2502 for use as a contraceptive device is provided with a wire reinforcement. The reinforcement wire may assist in the insertion of the encapsulation device 2502 along the vaginal canal using, for example, a scope or a catheter. In one aspect, the wall thickness of the encapsulation device may range approximately between 0.004 inches to 0.02 inches. The encapsulation device 2502 may also comprise a thicker wall ePTFE that has sufficient column strength to track through the vaginal canal with a scope. However, a relatively thicker encapsulation device, for example, at wall thickness of approximately 0.05 inches or more, may not benefit from the wire reinforcement, and thus, such reinforcement may not be used.

In yet a further embodiment, the encapsulation device 2502 for use as a contraceptive device may also include a temporary wire running down its middle. This would further aid in the delivery of the encapsulation device 2502 to the desired location. After deployment to the target location, the temporary wire may be removed through an injection port of the catheter, such as lumen catheter 2401. In one aspect, the temporary wire may be sufficiently long to run through the entire length of the catheter 2401. When the encapsulation device 2502 is at the desired location, the wire is then pulled out of the catheter 2401 and discarded. Moreover, the encapsulation device 2502 may also include a cap portion formed from a cut in the wall of the encapsulation membrane, or a completely separate end cap piece (see, for example, FIG. 4) bonded or molded to the encapsulation contraceptive device. The sealed end of the encapsulation contraceptive device may be pinched and bonded together with adhesive or heat, or may alternately, include a twisted end (see, for example, FIG. 2).

The encapsulation device of the present invention can also assist patients with atrial fibrillation (AF). Attached to the left atrium of the heart is a small pocket called an appendage (also called an auricle). This appendage expands and contracts just like the rest of the heart, causing blood to flow in and out, if the heart is normal and healthy. During atrial fibrillation (AF), the heart can beat irregularly and/or too fast. When this occurs, the appendage does not force blood out, and the blood can form clots which can eventually dislodge and cause a stroke, or other ailments.

Patients with atrial fibrillation (AF) typically have this condition as result of the Left Atrial Appendage (LAA) not having a valve mechanism to block off blood flow. The encapsulation device discussed herein in conjunction with FIGS. 27-33 is configured to unfold similar in mechanism to car sun visors in automobiles. In this manner, within the scope of the present invention, the porous encapsulation device that is configured to weep adhesive fluid or that uses mechanical anchors, may be used to occlude a diseased left atrial appendage.

FIG. 27 illustrates an atrial appendage encapsulation device 2701 with a separate injection port in accordance with one embodiment of the present invention. As shown, the encapsulation device which is delivered to the target location using a catheter (not shown) 2701 includes an injection port 2702 through which fluid is delivered to the encapsulation device 2701. The cut away section in FIG. 27 is provided for purposes of illustrating the hollow cavity of the encapsulation device 2701. Other applications of the encapsulation device in addition to treating atrial appendage includes, but is not limited to contraception application, vertebral body reconstruction, as a AAA device, and sphincter reconstruction.

FIG. 28 is the ePTFE encapsulation device shown in FIG. 27 with wire reinforcement in accordance with one embodiment of the present invention. As shown, the encapsulation device 2701 includes wire reinforcement 2801 to provide physical support in, for example, expansion of the encapsulation device 2701. More specifically, the NiTi support wire 2801 is provided in the encapsulation device 2701 to assist in the expansion of the encapsulation device 2701 prior to being filled with the displacement fluid once it is delivered out of the delivery catheter (not shown).

Also shown in the Figure are anchor wires 2802 attached to the encapsulation device 2701 to further secure the encapsulation device 2701 in the desired location. More specifically, the anchor wires 2802 are configured to penetrate into the tissue wall of the vessel of artery in which the encapsulation device is deployed to secure the device in the deployed location. In one embodiment, the length of the anchor wires 2802 are approximately 2 mm so as not to puncture through the vessel or artery wall. The anchor wires 2802 may be soldered to the one long wire (for example, the wire reinforcement 701 shown in FIG. 7), or alternatively, may be bonded to the one long wire with a resistance weld bonding technique, or adhesive. In one embodiment, the hypodermic needle tubing may be cut in such a way to create the anchor wires 2802. More specifically, first cut and discard a 2 mm long by 180 degree section away from a 4 mm long hypo tube. Then, bend the 180 degree half of the tube still attached part out, and slide the non cut part over the wire and bond in place with cyanoacrylate. This procedure is repeated until there are as many anchor wires as desired. It should be noted that those skilled in the art would appreciate that there are many different ways to create anchors on a wire.

FIG. 29 is an encapsulation device 2901 for use in treating atrial appendage in accordance with another embodiment of the present invention. Referring to FIG. 29, the encapsulation device 2901 is physically shaped substantially as a sphere. Again, the cut away section is illustrated in FIG. 29 to show the inner cavity of the encapsulation device 2901.

FIGS. 30-33 illustrate the application of the encapsulation device using a guide catheter in the left atrium of a heart for treating diseased atrial appendage in accordance with one embodiment. More specifically, it can be seen from FIG. 30 that a guide catheter is 3001 is introduced into the left atrium of the heart. FIG. 31 illustrates the encapsulation device 3002 advanced through the guide catheter shown in FIG. 30, and FIG. 32 illustrates the encapsulation device 3002 shown in FIG. 31 filled with displacement fluid through the injection port 3003 of the guide catheter 3001 in accordance with one embodiment. As before, the encapsulation device 3002 which may be comprised of ePTFE filled with saline solution and contrast solution first to ensure it is in the correct location in the left atrium. When it is determined that the encapsulation device 3002 is in the desired location in the left atrium, adhesive is injected as the displacement fluid via the injection port 3003 of the guide catheter 3001, causing the saline solution to be displaced out of the encapsulation device 3002 by a weeping procedure.

Finally, FIG. 33 illustrates the encapsulation device 3002 secured at the target location with the guide catheter 3001 removed in accordance with one embodiment. As can be seen from the Figure, the guide catheter 3001 with the injection port 3003 are removed and the encapsulation device 3002 is bonded in place within the desired location in the left atrium. It should be noted that once the guide catheter 3001 is removed, the encapsulation device 3002 is completely sealed off (where the injection port 3003 was engaged with the encapsulation device 3002) such that once in place in the LAA, the surface of the encapsulation device 3002 which comes into contact with the blood in the heart is non-porous, thus avoiding the possibility of thrombosis.

FIGS. 34A-34F illustrate an LAA low density center encapsulation device for use in atrial appendage application in accordance with another embodiment of the present invention. In this embodiment, the encapsulation device has a lighter weight or a lower density core. More specifically, FIG. 34A illustrates an injection port 3402 (of the guide catheter) with an encapsulation device 3401 mounted thereon. FIG. 34B illustrates a PET balloon 3403 with mold release on it or hydrophilic coating inserted into the encapsulation device 3401. FIG. 34C illustrates the inflating of the PET balloon 3403 with saline solution via injection port 3402, and FIG. 34D shows the inflated PET balloon 3403 surrounded by the encapsulation seal device 3401 which is subsequently filled with hardening and weeping adhesive solution (displacement fluid). Because of the coating on the PET balloon 3403, the hardening adhesive solution introduced into the encapsulation device 3401 will not stick to the PET balloon 3403. Thereafter, the saline solution is displaced with SPH or a spongy, low density material that is injected into the inner PET balloon 3403. Thus, as shown in FIG. 34E, the cavity left by the deflated and removed balloon is filled with SPH or another lower density material such as silicone. Then, as shown in FIG. 34F, the lower density core of the encapsulation device 3401 either touches the adhesive or sits inside a PET balloon inside the encapsulation.

Indeed, an encapsulation device that is too heavy may cause problems in a beating heart due to its weight. In the manner shown by FIGS. 34A-34F, there is provided a way of making the core of the encapsulation device have a lower density than the rest of the encapsulation device by, for example, having the center held open with a hard PET balloon that is then removed and replaced with a lower density material, for example, SPH.

FIG. 35 is an encapsulation device 3501 for use in a vertebral plasty application in accordance with one embodiment of the present invention. The encapsulation device 3501 may comprise ePTFE or PET and structurally may be configured to be shorter and wider than the encapsulation device for the contraceptive application discussed above in conjunction with FIGS. 22-26. The cutaway section of the Figure is provided to illustrate the hollow cavity of the encapsulation device 3501.

FIGS. 36-42 illustrate the encapsulation device 3501 for use in treating vertebral plasty in accordance with one embodiment of the present invention. FIG. 36, illustrates a bone 3601 with a cavity 3602. As shown in FIG. 37, a guide catheter 3701 is introduced into the bone 3601 such that the guide catheter 3701 is within the cavity 3602 of the bone 3601. In application, an encapsulation device 3801 (for example, comprising ePTFE material) of FIG. 35, for example, is mounted to an injection port 3802 of the guide catheter 3701 and provided into the cavity 3602 of the bone 3601, as shown in FIG. 38. Thereafter, as shown in FIG. 39, fluid is introduced into the encapsulation device 3801 through the injection port 3802 via the guide catheter 3701 to expand the encapsulation device 3501 within the cavity 3602 of the bone 3601. Responsive to the expansion of the encapsulation device 3801 in the cavity 3602, the bone 3601 itself also expands in the direction shown by the arrow A in FIG. 40 until the cavity 3602 in the bone 3601 is completely filled with the expanded encapsulation device 3801. Thereafter, when the cavity 3602 is completely filled with the expanded encapsulation device 3801, the adhesive injected into the encapsulation device 3801 via the injection port 3802 is cured to harden the expanded encapsulation device 3801, as shown in FIG. 41, and the guide catheter 3701 along with the injection port 3802 is removed, as shown in FIG. 42.

At this point, it can be seen that the encapsulation device 3801 as well as the bone 3601 are fully expanded. Furthermore, as discussed above, the adhesive which slightly weeps through the walls of the encapsulation device 3801 is configured to bond it in place. Also, it should be noted that the removal of the guide catheter 3701 seals the encapsulation device 3801 at the end where it was mounted to the injection port 3802. With both ends of the encapsulation device 3801 sealed, the cavity 3602 is completely filled with the encapsulation device 3801.

FIG. 43 illustrates a magnified view of the guide catheter 3701 with the injection port for use with the encapsulation device of FIG. 35 in a vertebral plasty application in accordance with one embodiment of the present invention. As can be seen from the Figure, the adhesive or other displacement fluid is injected into the encapsulation device 3801 via the injection port 3802 of the guide catheter 3701.

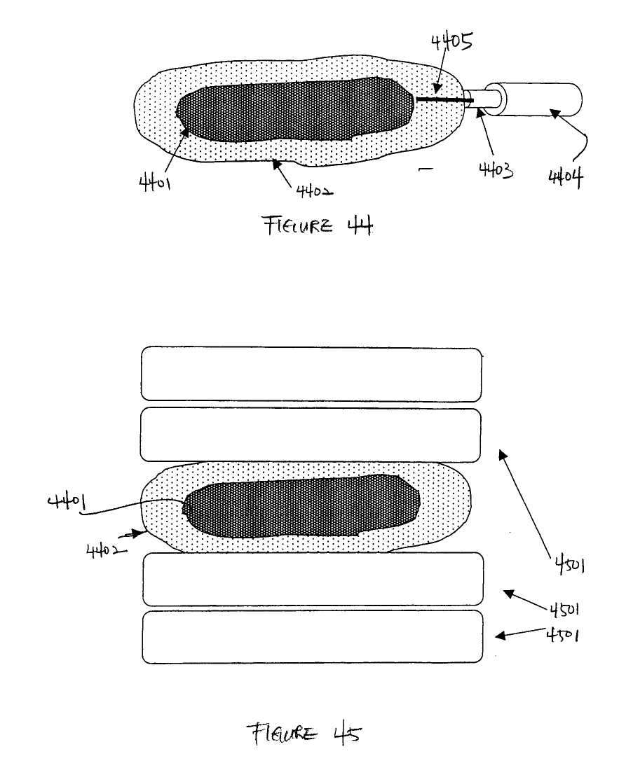

FIG. 44 illustrates an encapsulation device for use as a vertebral plasty double balloon in accordance with one embodiment of the present invention. As shown, there is provided a harder core or inner encapsulation portion 4401, and a softer outer encapsulation portion 4402. Also shown in the Figure are a guide catheter 4404, a first injection port 4403 for the outer encapsulation device portion 4402, and a second injection port 4405 for the inner encapsulation portion 4401. In this manner, the multi-layered encapsulation device (4401, 4402) may be configured with two injection ports 4403, 4405, each with the self sealing valves.

In application, the inner encapsulation portion 4401 is first filled with the displacement fluid, and the corresponding second injection port 4405 for the inner encapsulation portion 4401 is removed. Thereafter, the outer encapsulation portion 4402 is filled via the first injection port 4403 with the displacement fluid. In one embodiment, the inner encapsulation portion 4401 balloon is comprised of a substantially non-porous material, while the outer encapsulation portion 4402 is comprised of a substantially porous material. Accordingly, the inner encapsulation portion 4401 is configured to be harder as compared with the outer encapsulation portion 4401, configured for absorbing pressure. Indeed, as shown in FIG. 45, the multi-layered encapsulation device (for example, comprising encapsulation portions 4401, 4402) may be suitably configured to provide support between spinal discs 4501. More specifically, within the scope of the present invention, either or both of the multi-layered encapsulation portions 4401, 4402 may be non-porous, the innermost layer of the encapsulation device may be configured to be the hardest layer (or softest layer), and also, the encapsulation device may include more than two balloons (or encapsulation portions) of various hardness. The encapsulation approach discussed herein may also be used to aid in the containment of the nucleus pulposus prior to intervertebral herniated (ruptured) disc, by being placed in the annulus fibrosis in accordance with the procedure shown in the corresponding figures.

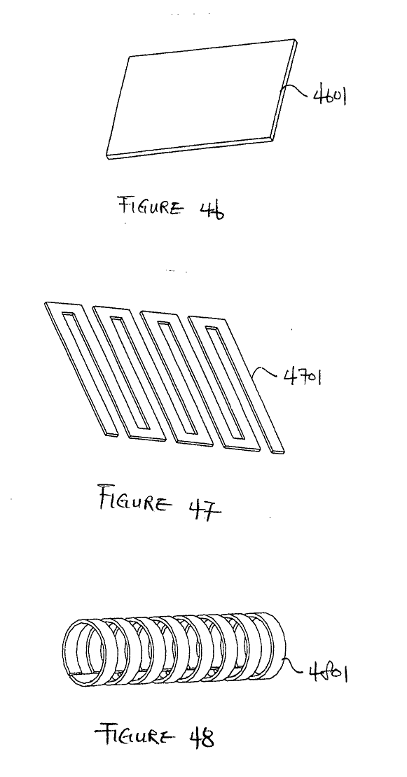

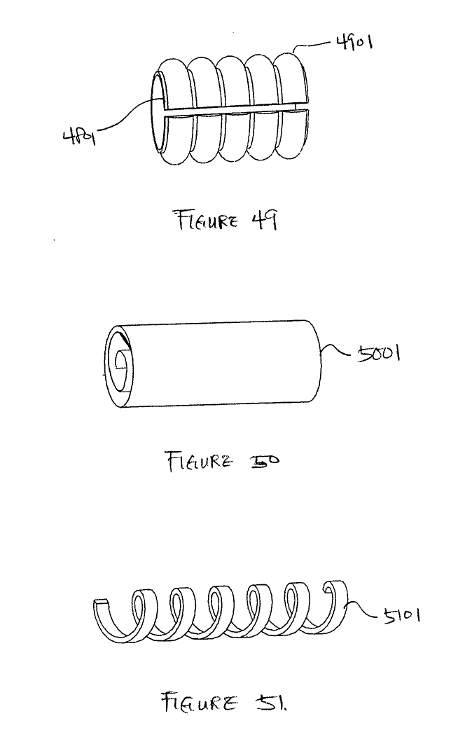

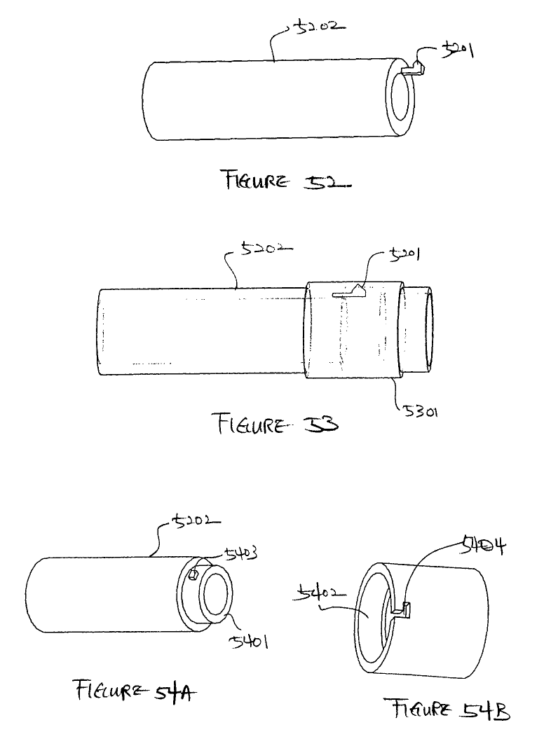

FIG. 46 illustrates a flat NiTi sheet 4601 for use in a stent-like application in accordance with one embodiment of the present invention. With the flat NiTi sheet shown in FIG. 46, a predetermined shape 4701 may be cut or etched therefrom, as shown in FIG. 47. In this manner, construction of the stent may begin as a flat NiTi sheet 4601 (FIG. 46) which is then cut or etched to the pattern shown in FIG. 47. Thereafter, the cut or etched NiTi sheet shown in FIG. 47 may be rolled into a tubular shape 4801 or heat shaped as shown in FIG. 48. Thereafter, the cut or etched NiTi sheet rolled into a tubular shape may be covered with porous material such as ePTFE 4901 as shown in FIG. 49. For example, the shaped NiTi sheet is covered with two sheets ePTFE material. In one embodiment, the inner ePTFE sheet is configured to be slightly thicker (e.g., 0.02 inches, approximately), and thus will provide some rigidity. The outer sheet of ePTFE material which is configured to expand out is configured to be thinner, such that adhesive may weep out of the walls of the outer sheet. For purposes of deployment to target location, the NiTi sheet 5001 may be folded or rolled up for delivery by a catheter (not shown) as shown in FIG. 50.

FIG. 51 illustrates a NiTi ribbon material shaped into a helix for use as a support member in a tubular encapsulation device in accordance with one embodiment of the present invention. As shown, in one aspect, the NiTi material may comprise approximately 0.002 to 0.02 inches in thickness, shaped into a helix 5101. The helix shaped support member 5101 may be deployed inside a tubular encapsulation device, and also, may be used as a support member in an implantable encapsulation device. More specifically, it may be wrapped down inside a sheath (not shown) and configured to pop open like a stent when pushed out of the sheath, such that the encapsulation device will expand at the desired location, and thereafter, the subsequent injection of the hardening displacement fluid bonds the encapsulation device in place.

FIG. 52 illustrates a single lumen injection port with a cutting member for use with the encapsulation device in accordance with one embodiment of the present invention. As shown, there is provided a cutting member 5201 at one end of the injection port 5202 (of a catheter, for example). The single lumen injection port 5202 may be made of 63 durometer PEBAX (family of nylons) with an approximate 0.01 inch wall, while the cutting member 5201 may be made of stainless steel or hard plastic. In one embodiment, the 0.01 inch PEBAX may have a stainless steel braid reinforcement (not shown) in its wall giving it good torque characteristics. The stainless steel braid may be, for example, a 16-wire, 80 picks per inch, 0.001×0.005 inch, type 304V stainless steel braid. Once the single lumen injection port 5202 is in the proper location and the encapsulation device (not shown here) is filled, the stainless steel braid reinforced port is rotated. This movement would sever the thin-walled low durometer tube and detach the encapsulation device.

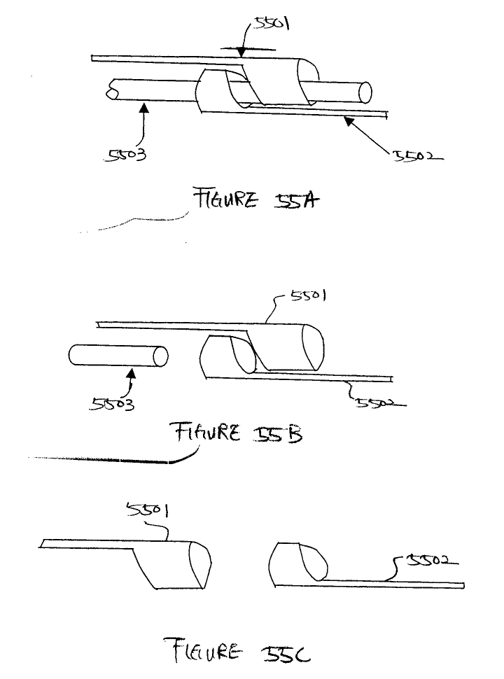

FIG. 53A illustrates the injection tube of FIG. 52 with the cutting member to engage with the encapsulation device, and FIG. 53B illustrates the injection tube of FIG. 52 after the cutting member has engaged with the encapsulation device in accordance with one embodiment of the present invention. Referring to the Figures, the encapsulation device 5301 shown engages with the cutting member 5201 of the injection port 5202, which may be rotated (e.g., manually) to cut the encapsulation device. Also shown in the Figure is a thin walled PEBAX which has been shrunk down with heat shrink tubing.

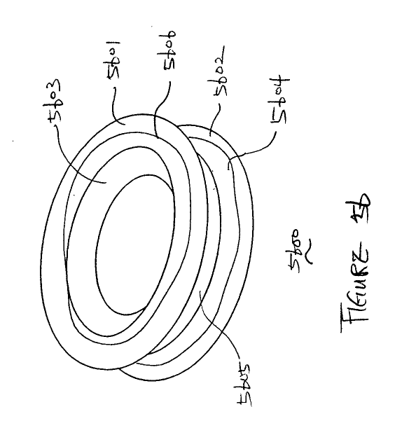

FIGS. 54A-54B illustrate male and female portions, respectively, of an encapsulation device injection valve in accordance with one embodiment of the present invention. As shown in the Figures, the male portion 5401 of the injection port 5202 is configured to operatively couple to the respective female portion 5402 of the injection port 5202. More specifically, the male portion 5401 is configured to couple to a guide catheter (not shown) and the female portion 5402 is configured to couple to the encapsulation device (not shown). A male coupling section 5403 is provided on the male portion 5401 and is configured to engage (and thus lock in place) with the respective groove portion 5404 on the female portion 5402 of the injection port 520. After the injection of the displacement fluid into the encapsulation device (not shown), the male portion 5401 of the injection port 5202 is rotated and retracted from the groove portion 5404 in the female portion 5402 of the injection port 5202. Once the hardening fluid such as the adhesive displacement fluid has mostly cured into a gel state, for example, the injection catheter is removed and the displacement fluid is hardened sufficiently such that it does not leak out of the injection port 5202, thus effectively providing a self-sealing valve.

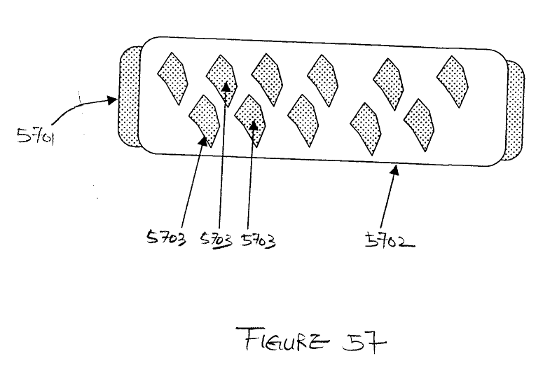

FIGS. 55A-55C illustrate the operation of an injection valve for use with the encapsulation device in accordance with another embodiment of the present invention. As shown in the Figures, the injection valve comprises two parts—an upper section 5501 configured to attach to the guide catheter (not shown), and a lower section 5502 which is configured to attach to the encapsulation device (not shown). As further shown in FIG. 55A, the injection port 5503 is provided such that it is engaged with both the upper section 5501 and the lower section 5502 of the valve. When the catheter is retracted, the upper section 5501 and the lower section 5501 are configured to physically separate. Indeed, when the injection port 5503 is retracted after the injection of the displacement fluid into the encapsulation device, the upper section 5501 and the lower section 5502 of the valve are configured to slide apart from each other. In one embodiment, the lower section 5502 of the valve is physically configured such that it is permanently attached to the encapsulation device even after the injection port 5503 is retracted, and thus, it stays in the body along with the encapsulation device.

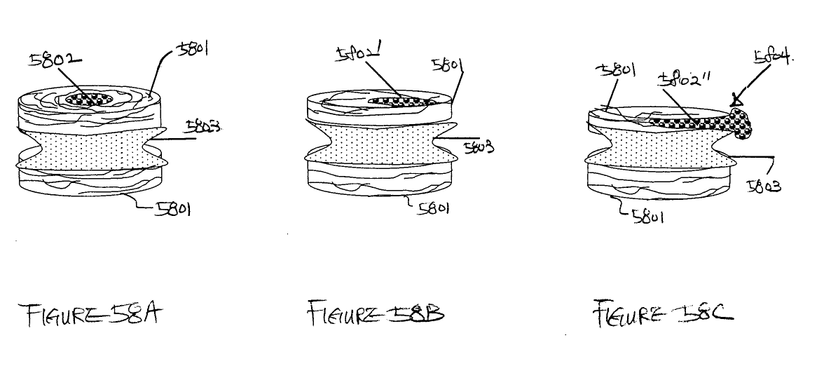

FIG. 56 is a three-dimensional perspective view of the encapsulation device 5600 in accordance with another embodiment of the present invention. The encapsulation device 5600 includes two rings 5601, 5602, comprised of ePTFE material filled with an adhesive such as a commercially available adhesive curable with ultraviolet (UV) light. For example, upon exposure to the appropriate light source (such as UV light source), the one part adhesive cures completely within seconds to form thermoset or thermoplastic polymers with the desired adhesion to a wide variety of substrates. Typical curing time period may range between two to sixty seconds.

Referring back to FIG. 56, the ring 5601 has a membrane 5603, and a ring 5602 has a membrane 5604, which join in the center to provide a common membrane 5605 sealing the hole within the rings. Each ring and associated membrane may be assembled from a sheet of ePTFE material having a solid disc shape. The outer edge of the disc is folded over and attached to itself to form the ring. The rings would further include a valve to permit filling of the rings. It is anticipated that the encapsulation device 5600 be deployed in an unfilled configuration, and the rings would be filled after deployment to secure it in place within a septal defect.

Also shown in FIG. 56 are NiTi support wires 5606 configured to assist the positioning of the encapsulation device 5600 in the septal defect prior to the injection of the displacement fluid. In one aspect, the encapsulation device is made out of an ePTFE material sheet that is cut into a circle and then folded over itself to form two opposing discs. Further, as discussed above, the encapsulation device 5600 may also include NiTi wire reinforcement 5606 inside the encapsulation rings to aid in the deployment of the device prior to filling. Within the scope of the present invention, any of the valves discussed above may be used with this encapsulation device 5600. The hardening solution inside the encapsulation device 5600 may displace saline solution previously injected. If the encapsulation device 5600 is not in the proper location, then it can be deflated and moved until it is positioned at the target location. Once it is in the proper location, the encapsulation device 5600 is filled with the displacement fluid using, for example, a guide catheter (not shown), and the displacement fluid hardens therein.

FIG. 57 illustrates a dual layer encapsulation device in accordance with one embodiment of the present invention. Referring to the Figure, a porous shell layer 5702 is provided around the outer surface of an encapsulation device 5701 which may be, for example, of ePTFE material. The porous shell layer 5702 may include a porous PET shell or a metallic tube having laser drilled holes thereon as discussed above. Referring back to the Figure, the holes 5703 on the porous shell layer 5702 in one embodiment are configured to allow adhesive or other fluid to weep therethough.

As discussed above, the dual layer encapsulation device in accordance with one embodiment of the present invention is particularly suitable for applications which require a very thin walled encapsulation device 5701 such that the outer porous shell layer 5702 may provide additional margin of safety.

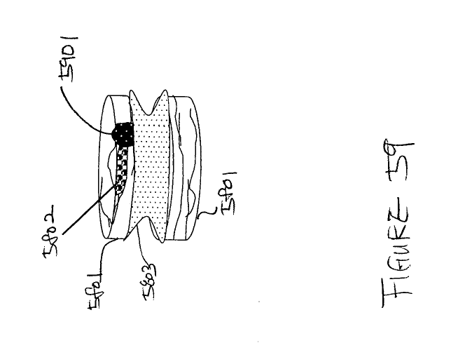

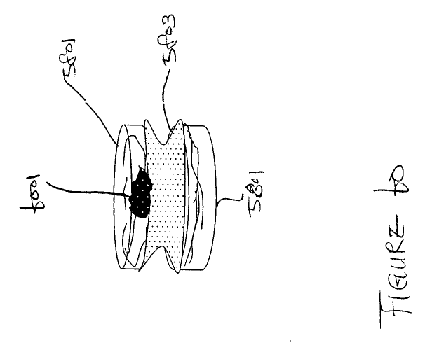

FIGS. 58A-58C illustrate the process of lumbar vertebrae degeneration commonly referred to as herniated disk. Referring to FIG. 58A, spinal disks include annulus fibrosis 5801 and nucleus pulposus 5802 which are positioned between the vertebral bones 5803 in the spinal column (where the spinal column contains vertebrae and disks between the vertebrae). More specifically, the nucleus pulposus 5802 comprises the center component of the spinal disk, and is substantially gelatinous, and functions as a cushion supporting the weight of the upper body. Over time with age, the nucleus pulposus 5802 will harden if it stays properly contained in the spinal disk. Referring back to FIG. 58A, the annulus fibrosis 5801 is made up of alternating collagen fibrils, and, as shown in the Figure, substantially surrounds the nucleus pulposus 5802.

Compression and/or pressure from supporting the weight of the upper body (for example, which exerts a force on the spinal column) can weaken the annulus fibrosis 5801 over time resulting in lumbar vertebrae degeneration. As a result of the weakened annulus fibrosis 5801, the compression on the spinal disk causes the nucleus pulposus 5802 to penetrate or extrude through the annulus fibrosis 5801 and rupture the nucleus pulposus 5802 resulting in a herniated disk. Indeed, referring to the Figures, the illustration of FIG. 58B shows the compression of the spinal disk which is resulting in the nucleus pulposus 5802′ to start to rupture, while the illustration of FIG. 58C shows the nucleus pulposus 5802″ which has ruptured through the surrounding annulus fibrosis 5801 at the point in the Figure shown with arrow 5804.

The nucleus pulposus 5802 can protrude out of the annulus fibrosis 5801 far enough to apply pressure and pinch nerve cells in the spine, or apply pressure on the spinal cord itself which causes severe pain, and in severe untreated cases, certain degrees of paralysis. While there exists treatment protocols for ruptured disks, none of the existing treatment presently available can effectively contain the nucleus pulposus 5802 when ruptured in the early stages. As discussed in further detail below, in accordance with one embodiment of the present invention, the encapsulation device may be used to effectively contain the nucleus pulposus 5802 within the annulus fibrosis 5801 surrounding the nucleus pulposus 5802, such that the early onset of herniated disk may be effectively treated before the symptoms become severe requiring a more aggressive treatment such as a spinal fusion procedure, or a complete spinal disk replacement.

FIG. 59 illustrates the encapsulation device as applied to treat ruptured spinal disks in accordance with one embodiment. Referring to the Figure, when the annulus fibrosis 5801 starts to degrade and become compressed, a hole may be drilled in the fibrous collagen tissue of the annulus fibrosis 5801 and the encapsulation device 5901 may be deployed via the drilled hole into the annulus fibrosis 5801 so as to prevent the rupturing of the nucleus pulposus 5802. In one embodiment, the encapsulation device 5901 may be substantially round in shape. However, within the scope of the present invention, the encapsulation device 5901 may be substantially tubular, or half-moon shaped, each of which may be effectively deployed into the drilled hole on the annulus fibrosis 5801 so as to prevent the rupturing of the nucleus pulposus 5802 resulting in herniated disk.

In one embodiment, saline solution may be used with the encapsulation device 5901 of the present invention which may be injected therein to accurately position the encapsulation device in the spinal disk. Moreover, the injected saline solution may be displaced with a secondary, permanent material as discussed in further detail above. In this case, the secondary material would displace the initially injected saline solution in the encapsulation device 5901, and thereafter may be cured to harden.

Alternatively, in accordance with another embodiment of the present invention, the encapsulation device 5901 may be injected with, for example, superporous hydrogel. Similarly, polymethylmethacrylate (PMMA) may be injected in lieu of the superporous hydrogel into the encapsulation device 5901. PMMA is commonly used as a bone cement, and hardens to a substantial strength when cured. Also, PMMA in an alternate embodiment may be blended with another material to soften it, as desired. Even further alternatively, the encapsulation device 5901 may be configured such that it comprises a porous side and a non-porous side, where the PMMA may be configured to weep through the porous side. In this case, the two porosity design of the encapsulation device 5901 may comprise two sheets bonded together as discussed above in conjunction with FIGS. 13 and 14. With the upper porous side design of the encapsulation device 5901, the PMMA may be weeped and bonded to the bone above, and not below the spinal disk, so that more patient flexibility may be provided as bonding two bones together is spinal fusion.

Furthermore, in one embodiment, a valve similar to that shown in FIGS. 2 and 27 may be used and which may be configured to be left behind in the body after deployment and detachment of the encapsulation device 5901. Additionally, an injection port similar to injection port 1901 shown in FIG. 19 (without the stent) may integrated with the encapsulation device 5901 to facilitate the injection of the displacement fluid and/or the saline solution.

In this manner, with early detection and diagnosis of back pain which is leading to the weakening process of the annulus fibrosis 5801, the encapsulation device in accordance with one embodiment of the present invention may be placed within the annulus fibrosis 5801 thus preventing the nucleus pulposus 5802 from penetrating through the annulus fibrosis 5801 and resulting in a ruptured spinal disk.

FIG. 60 illustrates the encapsulation device as applied to treat ruptured spinal disks in accordance with another embodiment. Referring to the Figure, the encapsulation device 6001 is configured to substantially replace the nucleus pulposus 5802, such that the encapsulation device substantially entirely fills the portion of the spinal disk within the annulus fibrosis 5801. In this manner, in the cases of early stages of spinal disk degeneration, the deployment of the encapsulation device 6001 to replace the nucleus pulposus 5802 would effectively provide the support for the spinal disk carrying the weight of the upper body.

In the manner described above, within the scope of the present invention, the encapsulation device may be made in a controlled manner with a broad range of porosity from no pores to vary large pores. More specifically, in one aspect, the encapsulation device may be adhered to itself in layers, in different three-dimensional shapes, and different components can be made to have different porosities and thus, configured to behave differently to suit the desired application such as, for example, by layering, by using different porosities, or by using components with different porosities—with one side porous and the other side, non-porous.

Furthermore, as discussed above, the encapsulation body may be filled with a bioactive or biocompatible agent, and also be optionally anchored in vivo. In addition, the encapsulation device may be filled with one or more fluids such hydrogels, SPH, or a caprolactone based polymer which foams with highly interconnected pore structures containing macropores and micropores. Under suitable conditions, for example, the caprolactone based polymer expands up to approximately 400% which assists a physician to properly deploy and position the encapsulation device at the desired location.

Additionally, the encapsulation device in a further embodiment may be doped with suitable material to render it more or less bioactive and/or radiopaque. Further, the encapsulation device within the scope of the present invention may include material other than ePTFE. Indeed, the weeping membrane of the encapsulation device may be made from any suitable porous material. By way of one example, a metal such as Nickel Titanium (NiTi) alloy (Nitinol) may be made porous by laser drilling small holes through its wall. Additionally, other examples of the encapsulation body material include metal/polymer mesh, perforated polymer/metal, and polyester fiberfill (also used as the material for endovascular prosthesis).

Various other modifications and alterations in the structure and method of operation of this invention will be apparent to those skilled in the art without departing from the scope and spirit of the invention. Although the invention has been described in connection with specific preferred embodiments, it should be understood that the invention as claimed should not be unduly limited to such specific embodiments. It is intended that the following claims define the scope of the present invention and that structures and methods within the scope of these claims and their equivalents be covered thereby.

1. An encapsulation device, comprising:
an expandable, porous body having a cavity therein, the body having a sealed end and a sealable end, wherein the body is configured to receive one or more fluids through a port in the sealable end, wherein the body is configured to expand to conform to a shape of a target, and wherein the sealable end may be sealed to prevent leakage into the body.

2. The device of claim 1 wherein said body comprises one of expanded Polytetrafluoroethylene (ePTFE), a porous membrane, a polyester fiberfill, a metal mesh, a polymer mesh, a perforated polymer, and a perforated and/or porous metal.

3. The device of claim 1, wherein the target comprises a location within the human body.

4. The device of claim 1, wherein the port in the sealable end receives a first fluid into said cavity to expand the body to conform to the shape of the target, and wherein the port receives a second fluid into said cavity which displaces the first fluid by diffusing the first fluid through the pores in the body and which cures to secure the body to the target.

5. The device of claim 4, wherein the first fluid comprises a saline solution.

6. The device of claim 4, wherein the second fluid comprises one or more of an adhesive, a hydrogel, and a bioactive agent.

7. The device of claim 4, wherein the second fluid is more viscous than the first fluid.

8. The device of claim 4 wherein the second fluid is less viscous than the first fluid.

9. The device of claim 8 wherein said second fluid includes polyvinylpyrrolidone (PVP).

10. The device of claim 4 wherein the first fluid and the second fluid are radiopqaue.

11. The device of claim 1, wherein the body comprises metal having holes.

12. The device of claim 11, wherein the metal comprises nitinol.

13. The device of claim 1, wherein the port in the sealable end comprises a valve configured to open to receive one or more fluids and close to prevent leakage into the body.

14. The device of claim 1, wherein the body includes a wire reinforcement.

15. The device of claim 14 wherein said wire reinforcement includes a support wire positioned substantially within said body.

16. The device of claim 14 wherein said wire reinforcement includes a plurality of anchor wires positioned substantially around the outer surface of said body.

17. The device of claim 14, wherein the wire reinforcement comprises one or more of nitinol, stainless steel, and a structural polymer.

18. The device of claim 14, wherein the wire reinforcement is provided within the body.

19. The device of claim 1, wherein the body comprises a first membrane and a second membrane within the first membrane, wherein the second membrane is more porous than the first membrane.

20. The device of claim 1, wherein the body comprises a first membrane and a second membrane within the first membrane, wherein the first membrane is more porous than the second membrane.

21. An encapsulation device, comprising:
an expandable, porous body having a cavity therein, the body having a sealed end and a sealable end, wherein the body is configured to receive one or more fluids through a port in the sealable end, wherein the body is configured to expand to conform to a shape of a target, wherein the sealable end may be sealed to prevent leakage into the body, wherein said body comprises expanded Polytetrafluoroethylene (ePTFE), and further, wherein the port in the sealable end receives a first fluid into said cavity to expand the body to conform to the shape of the target, and wherein the port receives a second fluid into said cavity which displaces the first fluid by diffusing the first fluid through the pores in the body and which cures to secure the body to the target.

22. The device of claim 21, wherein the target comprises a location within the human body.

23. The device of claim 21, wherein the first fluid comprises a saline solution.

24. The device of claim 21, wherein the second fluid comprises an adhesive.

25. The device of claim 21, wherein the second fluid is more viscous than the first fluid.

26. The device of claim 21, wherein the body comprises metal having holes.

27. The device of claim 26, wherein the metal comprises Nickel Titanium alloy (Nitinol).

28. The device of claim 21, wherein the port in the sealable end comprises a valve configured to open to receive one or more fluids and close to prevent leakage into the body.

29. The device of claim 21, wherein the body comprises a wire reinforcement.

30. The device of claim 29, wherein the wire reinforcement comprises one or more of Nitinol, stainless steel, and a structural polymer.

31. The device of claim 29, wherein the wire reinforcement is provided within the body.

32. The device of claim 21, wherein the body comprises a first membrane and a second membrane within the first membrane, wherein the second membrane is more porous than the first membrane.

33. The device of claim 21, wherein the body comprises a first membrane and a second membrane within the first membrane, wherein the first membrane is more porous than the second membrane.

34. A method of providing an encapsulation device to a desired location, the method comprising:
expanding a porous body to conform to a shape of a target by introducing a first fluid into an opening in the body;
introducing a second fluid into the porous body to displace the first fluid through the porous body; and
allowing the second fluid to cure to secure the porous body to the target.

35. The method of claim 34, further comprising the step of inserting a wire reinforcement into the porous body.

36. The method of claim 35, further comprising the step of securing the wire reinforcement to the interior of the porous body.

37. The method of claim 35, further comprising the step of removing the wire reinforcement from the porous body.

38. The method of claim 34, wherein the body comprises one or more of expanded Polytetrafluoroethylene (ePTFE), porous Polyethylene Terephthalate (PET), and metal with holes formed therein.

39. The method of claim 34, further comprising the step of introducing the body to a location of the target.

40. The method of claim 34, wherein the target comprises a location within the human body.

41. The method of claim 34, wherein the first fluid comprises a saline solution.

42. The method of claim 34, wherein the second fluid comprises an adhesive.

43. The method of claim 34, wherein the second fluid is more viscous than the first fluid.

44. A method of providing an encapsulation device, the method comprising:
introducing a fluid into an opening of a porous body to conform the body to a shape of a cavity of a target location; and
curing the fluid introduced into the porous body at the target location such that the porous body substantially fills the cavity of said target location.

45. The method of claim 44 wherein the target location is in vivo.

46. The method of claim 44 wherein said fluid is a radiopaque agent.

47. The method of claim 44 wherein said fluid is a UV curable adhesive.

48. The method of claim 47 wherein said step of curing includes providing a UV light source to said adhesive.

49. The method of claim 44 wherein said porous body includes a plurality of pores, and further, wherein said introducing step includes the step of weeping at least a predetermined amount of said fluid out of said pores on said porous body at the target location.
